(19) United States
(12) Patent Application Publication
(10) Pub. No.: US 2026/0116112 A1
(43) Pub. Date: Apr. 30, 2026

(54) METHODS, SYSTEMS, APPARATUSES, AND DEVICES FOR FACILITATING CREATING LABELS FOR LABELING INTRAVENOUS INFUSION LINES

(71) Applicant: Vigilant Software, Trophy Club, TX (US)

(72) Inventor: John Fox Holt, Dallas, TX (US)

(73) Assignee: Vigilant Software, Trophy Club, TX (US)

(21) Appl. No.: 19/433,203

(22) Filed: Dec. 26, 2025

Related U.S. Application Data
(63) Continuation-in-part of application No. 18/037,778, now Pat. No. 12,537,093.
(60) Provisional application No. 63/242,671, filed on Sep. 10, 2021.

(51) Int. Cl. B41M 5/28; A61M 5/14; B41J 2/375; B41J 3/407; B41J 29/377; B41M 5/42; B41M 5/46; G06K 15/02; G09F 3/02; G09F 3/10

(52) U.S. Cl. CPC B41M 5/282; A61M 5/14; B41J 2/375; B41J 3/4075; B41J 29/377; B41M 5/42; B41M 5/46; G06K 15/024; G06K 15/028; G06K 15/1859; G09F 3/02; G09F 3/10; A61M 2205/60; B41M 2205/04; B41M 2205/38; G09F 2003/0201; G09F 2003/0202; G09F 2003/0208; G09F 2003/0211; G09F 2003/0226

(57) ABSTRACT

The present disclosure provides a method for creating a label for labeling an intravenous infusion line. Further, the method may include receiving an information associated with one or more intravenous infusion lines from input devices, analyzing the information, determining label content of labels associated with the intravenous infusion lines, determining label content formats of the label content, generating a label information for the labels based on the label content and the label content formats, and printing the label information on label sheets using a printing device based on the label information. Further, the printing device comprises a thermal printer configured for selectively inducing a chromatic transition in a thermochromic composition associated with the label sheets. Further, the printing of the label information on the label sheets is further based on the selective induction. Further, the printing of the label information creates the labels for the intravenous infusion lines.

### REFERENCE TO RELATED APPLICATIONS

This application is a continuation-in-part of U.S. patent application Ser. No. 18/037,778, titled “METHODS, SYSTEMS, APPARATUSES, AND DEVICES FOR FACILITATING CREATING LABELS FOR LABELING INTRAVENOUS INFUSION LINES”, filed on 19 May 2023, which is a national stage application, filed under 35 U.S.C. § 371, of International Patent Application No. PCT/US22/43185, titled “METHODS, SYSTEMS, APPARATUSES, AND DEVICES FOR FACILITATING CREATING LABELS FOR LABELING INTRAVENOUS INFUSION LINES” filed on 12 Sep. 2022, which claims the benefit of U.S. Provisional Ser. No. 63/242,671 , titled “SYSTEMS, METHODS, APPARATUS, AND DEVICES FOR CREATING LABELS FOR LABELING INTRAVENOUS INFUSION LINES”, filed 10 Sep. 2021, which are incorporated by reference herein in its entirety.

### FIELD OF THE INVENTION

Generally, the present disclosure relates to the field of data processing. More specifically, the present disclosure relates to methods, systems, apparatuses, and devices for facilitating creating labels for labeling intravenous infusion lines.

### BACKGROUND OF THE INVENTION

The present disclosure relates generally to the field of labeling technologies used in clinical environments, particularly those associated with the identification, tracking, and management of intravenous infusion lines and other time-sensitive or safety-critical medical conduits. The given field is of significant importance because accurate, timely, and unambiguous labeling directly affects patient safety, clinical workflow efficiency, medication-administration reliability, and compliance with institutional procedures. As modern healthcare settings continue to incorporate increasingly complex infusion therapies and simultaneous multi-drug regimens, the need for precise and highly legible label information has become correspondingly vital.

An objective that is desirable in the given field is to provide labeling solutions that may consistently convey essential clinical information in a manner that reduces cognitive load on healthcare staff, minimizes the likelihood of medication-related confusion, enables rapid identification of infusion characteristics, enhances traceability of tubing and line segments, and supports adherence to procedural and infection-prevention timelines. Achieving the given objective is crucial to minimizing clinical risk, avoiding unintended therapeutic outcomes, and improving overall care quality.

However, existing approaches for creating such labels may suffer from various limitations that hinder attainment of the aforementioned objective. For example, available labeling processes may require multiple manual steps, increasing the chances of transcription errors, miscalculations, or omissions of critical information. In some situations, labeling workflows may depend on separate materials, separate devices, or separate visual cues, making them difficult to standardize or customize according to clinical needs. In other scenarios, labels may lack sufficient visual differentiation to enable quick recognition of urgency indicators, timing information, or drug-specific warnings, contributing to delayed identification or misinterpretation. Some existing practices may also be insufficiently adaptable to dynamically changing infusion conditions, may not align well with high-acuity workflow demands, or may fail to integrate effectively with digital systems that generate, analyze, or communicate infusion-related information.

Additionally, current solutions may be constrained in their ability to support labeling tasks that rely on information derived from multiple sources, such as drug metadata, clinician identifiers, infusion-device parameters, or environmental conditions. As a result, clinical personnel may encounter difficulties when attempting to produce labels that accurately reflect real-time operational requirements, multi-parameter infusion characteristics, or compliance-related deadlines. The said limitations may also reduce the reliability of label-based cues used for identifying tubing segments, assessing expiration timings, or distinguishing between different categories of infusions.

Therefore, there is a need for improved methods, systems, apparatuses, and devices for facilitating creating labels for labeling intravenous infusion lines that may overcome one or more of the preceding problems.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a system for creating a label for labeling an intravenous infusion line. Further, the system may include a communication device which may be configured for receiving one or more information associated with one or more intravenous infusion lines from one or more input devices. Further, the system may include a processing device communicatively coupled with the communication device. Further, the processing device may be configured for analyzing the one or more information. Further, the processing device may be configured for determining one or more label contents of one or more labels associated with the one or more intravenous infusion lines based on the analyzing. Further, the processing device may be configured for determining one or more label content formats of the one or more label contents based on the determining of the one or more label contents. Further, the one or more label content formats includes a color code. Further, the color code includes seven different colors corresponding to seven days of a calendar week. Further, the processing device may be configured for generating one or more label information for the one or more labels based on the one or more label contents and the one or more label content formats. Further, the system may include a printing device communicatively coupled with the processing device. Further, the printing device may be configured for printing the one or more label information on one or more label sheets based on the generating of the one or more label information. Further, the printing device includes a thermal printer. Further, the one or more label sheets includes a base substrate with a thermochromic coating. Further, the thermochromic coating may be associated with two or more thermochromic compositions. Further, the one or more label sheets includes two or more sections. Further, the printing of the one or more label information further includes thermal printing of a required tubing change time alert on a section of the two or more sections. Further, the thermal printer may be configured for selectively inducing one or more chromatic transitions in one or more thermochromic compositions of the two or more thermochromic compositions. Further, the thermal printing of the required tubing change time alert on the section may be further based on the selective induction of the one or more chromatic transitions. Further, the thermal printing in the section color codes the required tubing change time alert with a color from the seven different colors representing a day of the seven days corresponding to the required tubing change time alert. Further, the printing of the one or more label information creates the one or more labels for the one or more intravenous infusion lines.

The present disclosure provides a method for creating a label for labeling an intravenous infusion line. Further, the method may include receiving, using a communication device, one or more information associated with one or more intravenous infusion lines from one or more input devices. Further, the method may include analyzing, using a processing device, the one or more information. Further, the method may include determining, using the processing device, one or more label contents of one or more labels associated with the one or more intravenous infusion lines based on the analyzing. Further, the method may include determining, using the processing device, one or more label content formats of the one or more label contents based on the determining of the one or more label contents. Further, the one or more label content formats includes a color code. Further, the color code includes seven different colors corresponding to seven days of a calendar week. Further, the method may include generating, using the processing device, one or more label information for the one or more labels based on the one or more label contents and the one or more label content formats. Further, the method may include printing, using a printing device, the one or more label information on one or more label sheets based on the generating of the one or more label information. Further, the printing device includes a thermal printer. Further, the one or more label sheets includes a base substrate with a thermochromic coating. Further, the thermochromic coating may be associated with two or more thermochromic compositions. Further, the one or more label sheets includes two or more sections. Further, the printing of the one or more label information further includes thermal printing of a required tubing change time alert on a section of the two or more sections. Further, the thermal printer may be configured for selectively inducing one or more chromatic transitions in one or more thermochromic compositions of the two or more thermochromic compositions. Further, the thermal printing of the required tubing change time alert on the section may be further based on the selective induction of the one or more chromatic transitions. Further, the thermal printing in the section color codes the required tubing change time alert with a color from the seven different colors representing a day of the seven days corresponding to the required tubing change time alert. Further, the printing of the one or more label information creates the one or more labels for the one or more intravenous infusion lines.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for creating labels for labeling intravenous infusion lines, in accordance with some embodiments.

FIG. 3 illustrates a block diagram of the system for creating a label for labeling an intravenous infusion line, in accordance with some embodiments.

FIG. 4 illustrates a block diagram of the system for creating a label for labeling the intravenous infusion line, in accordance with some embodiments.

FIG. 5 illustrates a block diagram of the system for creating a label for labeling the intravenous infusion line, in accordance with some embodiments.

FIG. 6 illustrates a block diagram of the system for creating a label for labeling the intravenous infusion line, in accordance with some embodiments.

FIG. 7 illustrates a flowchart of a method for creating a label for labeling an intravenous infusion line, in accordance with some embodiments.

FIG. 8 illustrates a flowchart of a method for creating a label for labeling an intravenous infusion line, in accordance with some embodiments.

FIG. 9 illustrates a flowchart of a method for creating a label for labeling an intravenous infusion line, in accordance with some embodiments.

FIG. 10 illustrates a flowchart of a method for creating a label for labeling an intravenous infusion line, in accordance with some embodiments.

FIG. 11 is a top perspective view of a device for creating labels for labeling intravenous infusion lines, in accordance with some embodiments.

FIG. 12 is a top perspective view of the device with the one or more input devices, in accordance with some embodiments.

FIG. 13 is a front view of the one or more label sheets, in accordance with some embodiments.

FIG. 14 is a front view of a label of the one or more labels, in accordance with some embodiments.

FIG. 15 is a flowchart of a method for creating labels for labeling intravenous infusion lines, in accordance with some embodiments.

FIG. 16 is a block diagram of a computing device for implementing the methods disclosed herein, in accordance with some embodiments.

FIG. 17 is a block diagram of a system for creating a label for labeling an intravenous infusion line, in accordance with some embodiments.

FIG. 18 is a block diagram of a system 1800 for creating labels for labeling intravenous infusion lines, in accordance with some embodiments.

FIG. 19 illustrates selective heating of one or more thermochromic layers 1906 from two or more thermochromic layers 1904 relative to one or more label sheets 1900 in accordance with some embodiments.

FIG. 20 illustrates selective heating of the one or more thermochromic regions 2004 from the two or more thermochromic regions 2002 relative to one or more label sheets 1900 in accordance with some embodiments.

FIG. 21 illustrates at least one sensor module 2102 included in the system 1800 for creating labels for labeling intravenous infusion lines, in accordance with some embodiments.

FIG. 22 illustrates at least one institution device 2202 included in the system 1800 for creating labels for labeling intravenous infusion lines, in accordance with some embodiments.

FIG. 23 illustrates a printhead unit 2302 included in the system 1800 for creating labels for labeling intravenous infusion lines, in accordance with some embodiments.

FIG. 24 illustrates a color-selection engine 2402 included in the system 1800 for creating labels for labeling intravenous infusion lines, in accordance with some embodiments.

FIG. 25A illustrates a flowchart of a method 2500 for creating a label for labeling an intravenous infusion line, in accordance with some embodiments.

FIG. 25B illustrates a continuation of the flowchart of the method 2500 for creating a label for labeling an intravenous infusion line, in accordance with some embodiments.

FIG. 26 illustrates a flowchart of a method 2600 for creating a label for labeling an intravenous infusion line including analyzing, using the processing device 1804, the at least one color palette information, in accordance with some embodiments.

FIG. 27 illustrates a flowchart of a method 2700 for creating a label for labeling an intravenous infusion line including determining, using the processing device, at least one print-path, in accordance with some embodiments.

FIG. 28 illustrates a flowchart of a method 2800 for labeling an intravenous infusion line, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems, methods, apparatus, and devices for creating labels for labeling intravenous infusion lines, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g., initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Glossary

In some embodiments, a communication device is a hardware device or an electronic device that is capable of transmitting an analog or digital signal over communication channels such as telephone lines, other communication wires, or wirelessly. An example of a communication device is a computer modem, which converts a computer's digital information to an analog signal for transmission over a telephone line. Further, the communication device may be a communication interface and/or a computing device with a communication connection.

In some embodiments, the communication device may be an electronic device that can be used for data and/or signal transmission and reception.

In some embodiments, an intravenous infusion line may be a soft, flexible tube placed inside a vein, usually in the hand or arm. Further, health care providers may use the intravenous infusion line to give a person and/or a user medicine or fluids intravenously.

In some embodiments, at least one information may include user information associated with the user of the intravenous infusion line, medicine information associated with medicines delivered using the intravenous infusion line, doctor information associated with doctors that prescribed the medicine which is delivered to the user using the intravenous infusion line, etc. Further, the user information may include user identifiers such as name, age, sex, disease, etc. Further, the medicine information may include medicine's names, medicine prescribed dosages, medicine expiry dates, medicine usage conditions, medicine storage conditions (storage temperature), etc.

In some embodiments, an input device may be a piece of equipment or an electronic device used to provide data and control signals to an information processing system, such as a computer, a computing device, or an information appliance.

In some embodiments, the input device may include at least one of an optical scanner, an RFID reader, and the computing device.

In some embodiments, the optical scanner may include a device found within most computer scanners that capture visual information and translate the visual information (image) into digital information that the computer is capable of understanding and displaying.

In some embodiments, the optical scanner is configured for scanning at least one visual representation of the at least one information present on at least one physical object.

In some embodiments, the radio-frequency identification (RFID) reader uses electromagnetic fields to automatically identify/detect and track tags attached to objects.

Further, the tags may include information about the objects.

In some embodiments, the RFID reader may include a radio transponder, a radio receiver, and a transmitter.

In some embodiments, at least one label content format may include a color code for one or more label content associated with a label. Further, the color code may include at least seven different colors corresponding to seven days of a calendar week. Further, the at least one label content format may include a position, an arrangement, a size, a color, a texture, etc. of each of the one or more label content of the label.

In some embodiments, one or more label sheets may include a piece of paper attached to medicine bottles, infusion bottles, etc. comprising the one or more label content.

In some embodiments, one or more labels may include a primary label and one or more secondary labels.

In some embodiments, one or more secondary labels may include a piece of paper attached to a medicine bottle comprising a drug name and administration location information.

In some embodiments, a primary label may include a piece of paper attached to a medicine bottle comprising primary label content such as user's information, medicine information, time and date of administration, a required tubing change time for intravenous infusion, a drug-specific warning, and location of the infusion's administration.

In some embodiments, the one or more label content may include user information, medicine information, time and date of administration, required change time for the infusion tubing, drug-specific warnings, and location of the infusion's administration on a patient, etc.

In some embodiments, the one or more label content may include prescription labeling comprising a written, printed, or graphic matter upon any drugs or any of its containers, or accompanying such a drug. Further, the one or more label content may seek to identify drug contents and to state specific instructions or warnings for administration, storage, and disposal.

In some embodiments, a processing device is an electronic circuitry or an electronic device (processor, microprocessor, etc.) that executes instructions comprising a computer program. The processing device performs basic arithmetic, logic, controlling, and input/output (I/O) operations specified by the instructions in the program. Further, the processing device may be a processor and/or a processing unit.

In some embodiments, the processing device may be circuitry for determining the one or more label content and the at least one label content format of the one or more labels based on the one or more label content.

In some embodiments, a printing device may be a machine that makes a persistent representation of graphics or text, usually on paper. While most representation is human-readable and some are non-human-readable, bar code printers are an example of expanded use for printers. Different types of printers include 3D printers, inkjet printers, laser printers, and thermal printers.

In some embodiments, the printing device may include a printer such as a thermal printer, a laser printer, etc. configured for printing the one or more label information on the one or more label sheets.

Further, the thermal printer uses heat for printing. Further, the one or more label sheets may be a paper with a thermochromic coating which is known as a thermal paper. Further, the thermochromic coating of the paper turns to different colors with different textures when the thermochromic coating is heated at different temperatures by the thermal printer. Further, the one or more labels may be the one or more label sheets with the one or more label information printed on a first side of the one or more label sheets and a coating of an adhesive on a second side opposite to the first side of the one or more label sheets. Further, the adhesive allows attaching the second side of the one or more label sheets to the intravenous infusion line, medicine bottles, infusion bottles, etc.

Further, the one or more label information may be a visual representation of the label generated by the processing device.

In some embodiments, a drug database is an organized collection of data stored and accessed electronically. Further, the drug database may be stored on a file system, while large databases are hosted on computer clusters or cloud storage.

In some embodiments, a sensor is an electronic device that produces an output signal for the purpose of sensing a physical phenomenon.

In some embodiments, the sensor may be configured for generating a tracking data associated with a labeling activity.

In some embodiments, the tracking data may include information, generated by the sensor, associated with a labeling activity. Further, the labeling activity may include an act of scanning the at least one visual representation of the at least one information present on the at least one physical object, an act of generating the label information, an act of printing the label by printing the label information on the one or more label sheets, an act of attaching the label to the intravenous infusion line, the medicine bottle, infusion bottle, etc., etc. Further, the tracking data may include a location and a time for the labeling activity.

In some embodiments, a biometric sensor may be used to collect measurable biological characteristics (biometric signals) from a human being, which can then be used in conjunction with biometric recognition algorithms to perform automated person identification.

In some embodiments, the biometric sensor may be configured for generating biometric data of a label administrator.

In some embodiments, a biometric data may include information generated by the biometric sensor.

In some embodiments, an external device may include an electronic device configured for receiving the tracking data and the at least one information for auditing.

### Overview

The present disclosure describes systems, methods, apparatus, and devices for creating labels for labeling intravenous infusion lines. Further, the disclosed system provides rapid and effective means to create detailed infusion line labels in line with best practices, each label consisting of one or more adhesive stickers created at one time via a thermal printer. This, in turn, greatly reduces the time and effort to label intravenous infusion lines, while also ensuring consistency, legibility, and compliance of intravenous line labels and their contents. Additionally, the disclosed system contains a database of National Drug Codes (NDCs) as well as how long each NDC drug may be administered intravenously before the infusion's tubing must be changed. The disclosed system is programmed to calculate the date and/or time at which an infusion's tubing must be changed at the time of label creation and to display this information in the label's contents with or without color coding, with varying colors representing the days of the week. This reduces, if not eliminates, human error in calculating required infusion tubing change times.

The disclosed system prints an intravenous infusion line label when prompted to do so by the scan of a medication's barcode. This may be done with the use of a scanner connected to the device, or by a mobile computer or stationary computer running proprietary software, which is paired wired or wirelessly to the device. In the case of using a scanner, the device's user can also scan their own identification barcode before a label is printed to provide the device with the label administrator's information. In the case of using a mobile computer or stationary computer, the administrator provides the software with their information at log-in, which is maintained on the device and applied to any labels created via scan with this mobile device until the label administrator is logged out of the software. These and other unique features of the disclosed system and methods are discussed below and illustrated in the accompanying drawings.

Further, the disclosed system describes the printing of the label when the drug is not present due to an incomplete database. The disclosed system prompts the user/nurse/doctor to select the drug or the number of hours to expire so they can simply hand-write the drug name.

Further, some nursing units don't even scan but use this menu i.e. neonatal ICUs (NICUs) have a smaller subset of drugs used and have primarily compounded drugs (Compounded drugs often have barcodes specific to the patient so the drug code will not be in our database.

Further, the nurses use the system to print non-IV drug labels for tubes that are coming out of the patient such as chest tubes or foleys. These tubes are also changed based on time. These are selected in our menu similar to drugs not found when scanned.

Further, the disclosed system describes some IV lines that are connected to a previously implemented IV line (or primary line) via a Y port. These are called secondary or piggyback IV lines. Many hospitals change these IV lines within 24 hours overriding normal rules. i.e. Morphine will usually be changed in 96 hours if associated with a primary line but if it is associated with a secondary line then it needs to be changed in 24 hours. The reasoning is the Y port creates a higher potential for infection. The disclosed system needs to understand if it is a primary line therefore a barcode is affixed to the printer to accept that metadata first which distinguishes between the primary IV line and the secondary IV line. When the nurse needs a label for the secondary line, he/she scans that barcode and then the drug barcode. If it is a primary line, they simply scan the drug name.

Further, the printer of the disclosed system queries a hospital database after the barcode scan. Occasionally, the disclosed system doesn't have the drug barcode the nurse scans (For example in the case of compounded drugs). The disclosed system may query a database to get information on that drug to print the label(s).

Further, the disclosed system describes one of the labels that can be applied to a syringe (that is being used to add drugs to an IV bag) or dressing.

The disclosed system and method of use will be understood, both as to its structure and operation, from the accompanying drawings, taken in conjunction with the accompanying description. It should also be understood that the mixing and matching of features, elements, and/or functions of one embodiment may be incorporated into another embodiment as appropriate unless described otherwise.

Further, the present disclosure describes a labeling system. Further, the labeling system includes a printer either operably associated with a scanner conductively connected thereto via a cord or associated with a mobile or stationary computer which includes a scanner. The printer includes a top pivotally connected to a base configured to carry label sheets therein. During use, the user can pivot open the top to position the label sheets within a cavity formed by the base. The label sheets are fed through a printing device enclosed within the base and exit through an opening where they are accessible to the user.

The printer is further provided with a display carried on top and in viewing access to the user. In one contemplated embodiment, the display displays information related to creating labels e.g., staff information, medicine information, date, time, and so forth, in addition to displaying prompts (e.g., “Scan Drug NDC”) and information related to the device's setup and settings. In an alternative embodiment, the display is manipulated by the user to manually input information, for example, with the use of a touch screen. The display may also be configured to read fingerprints for security verification prior to use.

Further, the printer includes one or more ports that allow communication with a computer for preprogramming. Additionally, the printer in the contemplated embodiment includes a power cord that provides electrical energy to the printer via an electrical outlet.

One of the unique features believed characteristic of the disclosed system is the use of a scanner to obtain information for printing the labels on a sheet or the label sheet. This feature greatly reduces the time and effort to create medical labels for intravenous infusion lines and potentially eliminates human error in the process.

The scanner includes a handle integrally attached to a reader. During use, the user may hold the handle and scan, for example, a barcode associated with the user's identification and a barcode associated with the type of medicine being used, as indicated by a box. It should be understood that the majority of medicine bottles include barcodes adhered to the bottle, which provide important medical information. Thus, the box may represent a barcode directly from the medicine bottle.

Further, an intravenous infusion line label sheet includes multiple labels which are created concurrently and are adhesively bonded to the sheet. The primary label contains key information related to the infusion. One or more secondary labels intended for tagging the infusion tubing in multiple places may be printed concurrently with the primary label. During use, a plurality of label sheets may be placed in the cavity of the base and thereafter fed through a printer device and the drug and user information from a scanner is placed on individual labels via thermal printing. The user then removes labels from the sheet and adhesively adheres the labels to the intravenous infusion tubing. In some embodiments, the labels could remain blank or contain only prompts for information, on which the user could then handwrite the necessary information.

Further, the user's information, medicine information, time and date of administration, required change time for the infusion tubing, drug-specific warnings, and location of the infusion's administration on the patient can be printed on the labels via the printer. Specifically, the label contains the drug name in section, date of administration in section, time of administration in section, required tubing change time printed in section, color-coded required tubing change time alert in section, the user (administrator) information in the section, drug warning in section, and drug type information in the section. The label contains the drug name in the section and administration location information in the section.

Further, the present disclosure describes a process that begins with obtaining the user's information, which can be achieved via scanner, through input into a mobile computing device, a radio-frequency identification (RFID) badge, or the like, as indicated by a box. The disclosed system may maintain the user's information for some amount of time to negate the need to re-input the user's information at each label creation, thus saving time. After the user's information is received, the user scans a medicine barcode, as indicated by the box. The disclosed system then identifies the scanned drug's identity in its preprogrammed database, calculates the required tubing change time for intravenous infusion tubing with this drug, and prints labels on the label sheet, containing the user's information, drug information, and required intravenous infusion tubing change time, as indicated by the box.

Further, the present disclosure describes the use of a special sheet that contains 3 labels. The main label has 7 different colors that correspond to the day of the week. This helps ICU nurses look at IV lines to understand if they need to change the tubing. Additionally, 3 labels are created so the nurse can apply labels to different parts of the IV tube—above the pump, below the pump, and near insertion.

Another characteristic feature of the disclosed system is the ability to be networked and be operably associate with an external database and cloud service. Accordingly, the disclosed system may have the ability to be reprogrammed wirelessly, as well as communicate information on its activity wirelessly to an external database, program, and/or cloud service. The data obtained from the disclosed system in this way may be reviewed and/or analyzed by third parties. The ability to push updated software wirelessly to the disclosed system may further its ability to create accurate labels as new drugs are added to the FDA's NDC database and hospital-specific codes and their related information are updated. The ability to communicate information from the disclosed system to an external entity serves to enable compliance and labeling tracking, for example, by ICU nursing managers interested in tracking individual user's labeling activity. Further, the disclosed system may include additional transceivers and other devices to enable these features.

Further, the present disclosure describes a stand-alone programmable printer for creating labels for labeling intravenous lines.

Further, the present disclosure describes an app that connects to the printer via Bluetooth or network for creating labels for labeling intravenous lines.

Further, the present disclosure describes a desktop program or API plugin that sends the data to the printer via Bluetooth or network for creating labels for labeling intravenous lines.

Further, the present disclosure describes an automated labeling system that quickly creates compliant, easy-to-read intravenous infusion tube labels.

In some embodiments, the present disclosure describes a multi-colored label substrate for thermal printing. Further, the present disclosure relates to systems, methods, devices, and apparatuses for generating and printing labels for labeling intravenous (IV) infusion lines. More particularly, the disclosure relates to label sheets comprising multi-colored thermal substrates, configured to interact with thermal printers so as to visibly generate color-coded information including infusion-related alerts, tubing change warnings, day-of-week coding, drug-specific identifiers, priority indicators, and clinical-workflow status indicators by selectively activating thermochromic regions of different colors embedded within the substrate.

Further, although color-coding is critical for safety in IV medication administration, traditional color-coded label systems rely on pre-printed colored sections, requiring hospitals to maintain inventory of numerous label variants. Approaching limit flexibility when generating dynamic content such as tubing-change alerts, time-sensitive infusion warnings, or patient-specific customizations.

Further, thermochromic printing enables producing colored output without multi-ink systems, but current thermal label sheets generally contain single-color or monochrome substrates. The inability to dynamically activate multiple contrasting colors on a single label sheet restricts the effectiveness of alerts, clinical significance of color variations, and ability to meet different healthcare institutions' labeling conventions.

Further, the present disclosure discusses the need for multi-colored thermal substrates, usable within thermal printers, to allow selective color formation in multiple regions of a label sheet, without requiring multiple ribbons, colored cartridges, or multiple print passes.

In some embodiments, the present disclosure provides multi-layer or regionally-segmented thermal label sheets incorporating two or more thermochromic dye layers, each configured to react at different activation temperatures or pulse intensities produced by a thermal printhead.

Further, in some embodiments:
The label substrate includes multiple thermochromic regions (e.g., black, blue, yellow, orange).
The printing device (a thermal printer) is configured to activate different color regions by applying controlled temperature ranges, variable printhead pressures, or pulse durations.
The system generates labels where tubing-change alerts, day-of-week indicators, drug-specific warnings, infusion status icons, or other content appear in different colors on the same label, without pre-printed ink.

Further, the said aspect synergizes with the disclosed label's sections and color codes for tubing changes, particularly the seven-color weekly coding system (e.g., “the color code comprises seven different colors corresponding to seven days of a calendar week”).

Further, the given feature increases contrast, human visibility, machine readability, and enhances safety in IV workflows.

In some embodiments, the present disclosure describes the following multi-colored thermal label substrate architecture:

### Multilayer Structure

A label sheet may include:
Base substrate (paper, polymer film)
Adhesive layer
Two or more thermochromic dye layers, each exhibiting a different visible color when activated:
   a. Layer A—black activation at ˜65° C.
   b. Layer B—blue activation at ˜45° C.
   c. Layer C—yellow activation at ˜35° C.
   d. Layer D—orange activation at ˜75° C.

Further, layers may be spatially overlapping or arranged in stacked vertical layers with selective permeability to specific wavelengths or thermal energies.

### Regionally Segmented Structure

In some embodiments, label sheets have segmented color zones, each containing a thermochromic composition associated with a specific printed section of the infusion label:
“Drug name” section: black/blue.
“Tubing change alert” section: yellow/orange.
“Day-of-week” section: colored wedge region with seven possible color activations.
“Infusion location” or “line type” section: multicolored symbolic region.
“Barcode/QR code region”: black-only high-resolution area for machine readability.

### Differential Activation by Temperature

The thermal printer produces different colors by operating at controlled temperatures. For example:
Standard pass at 65° C.→Black.
Low-temp pass at 40-45° C.→Blue.
Very low pass at 30-35° C.→Yellow.
High-temp pulse at 75-80° C.→Orange.

In some embodiments, the present disclosure describes the following printing device enhancements. Further, the printing device (thermal printer) is updated to support:
Multi-pass, multi-temperature printing on the same sheet.
Printhead modules with heat-zone isolation to activate different colors in adjacent pixels.
Use of feedback sensors (infrared, resistance-based) to measure substrate temperature during printing.
Color-printing control algorithms integrated with the processing device.

In some embodiments, the disclosed system enables multiple contrasting alert colors on a single label using the multi-colored substrate. Examples:
A red or orange alert is thermally revealed when tubing change time is imminent (≤1 hr.).
A yellow alert is printed for mid-window warnings (3-4 hrs. remaining).
A blue or black alert is printed for informational messages.

In some embodiments, the disclosed system using multi-colored substrates simplifies printing, without pre-printed stock. Further, the disclosed system enables:
Full seven-color printing via selective thermal activation.
Different “color palettes” per institution.
Automatic color adjustment based on staff color-blindness profiles.

In some embodiments, the disclosed system incorporates thermochromic layers that may be reversible or irreversible. Further, the embodiments include:
Reversible layers for temporary alerts (e.g., infusion paused).
Permanent layers for legally required information (e.g., drug name, patient ID).

Further, in some embodiments, Barcodes/QR codes are printed in black while surrounding color-coded areas provide human-visible context (e.g., infusion urgency).

Further, the present disclosure describes the following clinical workflow enhancements enabled by multi-color substrate.
Color-coded line type identifiers
   Primary line→Blue indicator.
   Secondary line→Black indicator.
   Emergency bolus line→Orange indicator.
   Continuous infusion→Yellow background.

### Drug-Class-Based Dynamic Coloring

The system may colorize specific drug classes:
Antibiotics→Yellow patches.
Sedatives→Blue patches.
Vasopressors→Orange high-visibility warnings.
Hazardous drugs→Black diagonal thermal lines.

### Priority Coding System

A multi-colored thermochromic substrate enables:
STAT medications printed with orange/yellow flashing zones (activated by alternating heating cycles).
Scheduled infusions printed with subdued blue/gray.
Delayed start medications printed with partially activated zones.

### Environmental Condition Alerts

Some thermochromic compositions may change color with temperature exposure over time. Further, the present disclosure may include embodiments where:
The label changes color if exposed to temperatures exceeding safety ranges (e.g., medication requiring refrigeration).
Time-dependent diffusion of color indicates elapsed time since printing, acting as a visual timer.

In some embodiments, the present disclosure may describe the following alternative embodiments:

### Tri-Color Laminated Film

A film including three thermochromic layers separated by micro-insulating layers enabling selective activation.
Zoned Activation Micro Printing Grid

A pixel grid where:
Rows correspond to low-temperature activation dyes.
Columns correspond to high-temperature dyes.
Allowing pixel-by-pixel color placement.

### Microencapsulated Dye Beads

Each bead contains pigments that activate at different temperatures.
Thermal pulses rupture selected capsules.

### Refillable Multi-Color Thermal Substrate

A reusable label substrate mounted on a rigid card for long-term tubing management visualization.

In some embodiments, the processing device associated with the disclosed system may incorporate:
A color-selection engine determining which thermochromic layer to activate.
A print-path planning algorithm controlling heat pulses.
A color-blindness accommodation module adjusting contrast.
A regulatory compliance engine ensuring critical alerts use high-visibility colors.

Further, the present disclosure may describe the following examples:

### Example 1—Black/Blue Substrate

A two-layer label sheet:
Blue dye activates at 40° C.
Black dye activates at 70° C.

Allows printing:
Blue: day-of-week section.
Black: drug name+barcode.

### Example 2—Yellow/Orange/Black Substrate

Three layers:
Yellow: 35° C.
Orange: 55° C.
Black: 70° C.

Used to print:
Yellow tubing-change “6 hrs. remaining”.
Orange tubing-change “1 hr. remaining”.
Black drug info & user ID.

### Example 3—Multicolor Institution-Specific Templates

Hospitals may define:
Custom sets of colors for different floors or departments.
Pediatric vs adult protocols.
ICU vs surgical suite label patterns.

Further, the present disclosure describes the following advantages of the disclosed system over existing systems.
Multi-color output from a single thermal print pass.
No ink cartridges or color ribbons.
Reduced inventory of pre-colored labels.
Dramatically enhanced visibility and safety.
Better support for complex medical workflows.

Further, the disclosed multi-colored thermal label substrate system significantly expands the capabilities of the labeling system by providing dynamic, flexible, multi-color thermal printing for IV infusion line labeling, enabling improved safety, workflow efficiency, and visual clarity.

In some embodiments, the present disclosure describes the following techniques of operation associated with the disclosed system:

### 1. Temperature-Tiered Activation

Different thermochromic dye layers activate at unique temperature thresholds.
Low heat→yellow
Medium heat→blue
Higher heat→black
Very high heat→orange

Further, the printer modulates printhead temperature to reveal the desired color.

### 2. Pulse-Width Modulation

Color formation is controlled not only by temperature but also by the duration of heat application.
Short pulses activate shallow layers.
Long pulses penetrate deeper layers.

Further, the said aspect allows selective activation of stacked color layers.

### 3. Pressure-Modulated Color Activation

Certain thermal substrates respond differently under variable printhead pressure.
Higher pressure increases effective thermal transfer.
Lower pressure restricts heat diffusion.

Further, the pressure variation enables differential color formation.

### 4. Multi-Pass Sequential Printing

A printer may make multiple passes:
Low-temperature pass→activate color A.
Medium-temperature pass→activate color B.
High-temperature pass→activate color C.

Further, each pass adds new color information without disturbing previously activated zones.

### 5. Zoned Thermal Printhead Control

Printheads are divided into independently controlled micro-heating elements. Further, each pixel receives customized heat pulses. Further, adjacent pixels may activate entirely different colors, enabling pixel-level multi-color thermal images.

### 6. Layer-Selective Thermal Conductivity

Thermal substrates may use intermediate insulating layers with selective thermal permeability.
Low-temperature heat passes only to shallow layers.
High-temperature pulses penetrate deeper layers.
Enhancing layer separation and improves multi-color fidelity.

### 7. Microencapsulated Thermochromic Particles

The substrate contains microcapsules with pigments that activate when capsules rupture or melt at specific temperatures. Further, different capsule types produce different colors.

### 8. Chemical Threshold Activation

Some dyes rely on chemical activation thresholds rather than simple melting points. Further, phase-change materials trigger pigment development. Further, molecular rearrangement produces color shifts. Further, thermal input initiates the said transformations selectively.

### 9. Reversible vs. Irreversible Thermochromics

Substrates may incorporate both:
Reversible dyes (change color with temperature fluctuations).
Irreversible dyes (permanently activated).

Further, the said aspect allows mixed functionality such as permanent data plus temporary alerts.

### 10. Wavelength-Selective Thermal Absorption

Different pigments absorb infrared wavelengths differently. Further, thermal energy tuned to specific wavelength patterns may activate certain dyes preferentially.

### 11.Thermal Gradient Imaging

Heating occurs across a gradient rather than discrete levels. Further, colors emerge as a function of precise gradient position, enabling blended or shaded multi-color zones.

### 12. Conductive-Pathway Steering

The substrate includes conductive inks or pathways that channel heat to specific zones. Further, directing thermal current changes which regions activate.

### 13. Magnetically Enhanced Heating

Some substrates incorporate magnetic nanoparticles. Further, when exposed to magnetic induction heating, specific regions rise in temperature and activate selected colors.

### 14. Time-Dependent Diffusion Effects

Certain dyes continue to develop after heat is applied. Further, thermal printers exploit the said “after-image” effect to create gradient or dual-color outputs depending on timing.

### 15. Micro-Patterned Substrates

Substrate surface is patterned with micro-textures or micro-channels that alter heat flow. Further, the pattern selectively enhances or suppresses activation of certain colors.

### 16. Thermo-Pressure Phase Control

Color formation depends on combined heat +compression events. Further, specific combinations activate specific dyes, enabling combinatorial color creation.

In some embodiments, the present disclosure describes a method of printing a label with a direct thermal printer. Further, the disclosed method may include a step of providing a label substrate having a printable region. Further, the printable region comprising (i) a first thermochromic color-forming composition and (ii) a second thermochromic color-forming composition that spatially overlap within the printable region (stacked and/or intermixed). Further, the disclosed method may include a step of determining a target color for the printable region. Further, the disclosed method may include a step of selecting a thermal energy profile corresponding to the target color. Further, the disclosed method may include a step of driving a thermal printhead to apply the selected thermal energy profile to the same printable region. Further, the applying of a first thermal energy profile causes the printable region to exhibit a first color, and applying of a second thermal energy profile causes the printable region to exhibit a second, different color.

In some embodiments, the present disclosure describes a method of printing a label with a direct thermal printer. Further, the disclosed method may include a step of providing a substrate having, within a single printable region: a first thermochromic layer that develops a first color at/above a first activation threshold and a second thermochromic layer that develops a second color at/above a second activation threshold higher than the first. Further, the disclosed method may include a step of applying, to the same printable region, a thermal dose selected from at least {D1, D2}. Further, D1 activates the first layer without activating the second layer to display the first color, and D2 activates both layers so the region displays a resultant composite color different from the first color.

In some embodiments, the present disclosure describes a thermal printing system. Further, the thermal printing system may include a direct thermal printer including a thermal printhead. Further, the thermal printing system may include a controller. Further, the thermal printing system may include a memory storing a mapping between (i) a set of target colors and (ii) corresponding thermal energy profiles. Further, the thermal printing system may include a thermochromic label substrate with a printable region having multiple chromatic states. Further, the controller may be configured to receive print data specifying a target color for the printable region, select a thermal energy profile using the mapping, and drive the thermal printhead to apply the selected thermal energy profile to the printable region such that the same region exhibits the target color.

In some embodiments, the present disclosure describes a non-transitory computer-readable medium storing instructions that, when executed, cause a computing device to:
Generate a print job comprising, for each of a plurality of pixels, a coordinate (x, y) and an associated thermal dose value z selected from a plurality of non-binary dose values.
Translate the print job into printer control signals that drive a thermal printhead to apply pixel-wise thermal doses according to the z values. Further, the thermal doses cause a thermochromic printable region to exhibit different colors based on the z values.

In some embodiments, the present disclosure describes a method of calibrating a thermal printer for multi-color thermochromic label printing. Further, the disclosed method may include a step of printing a calibration pattern on a thermochromic substrate. Further, the calibration pattern may include a plurality of patches printed at different thermal dose levels. Further, the disclosed method may include a step of capturing, using an optical sensor, a measured color value for each patch after printing. Further, the disclosed method may include a step of generating a lookup table mapping dose levels to measured color values for the printer-substrate pair. Further, the disclosed method may include a step of subsequently printing a label by selecting a dose level based on the lookup table to cause a single printable region to exhibit a selected color.

Further, in some embodiments, the label substrate may include at least one printable region comprising two or more thermochromic color-forming compositions that are co-located within the same region (e.g., stacked layers and/or intermixed microcapsules), such that different thermal doses applied to the same region cause the region to display different colors.

In some embodiments, the present disclosure may provide a technologically advanced system, apparatus, substrate architecture, thermal-printing framework, and data-processing pipeline configured to generate intravenous infusion line labels containing multi-color, multi-format, multi-section content printed on a specialized multi-chromatic thermal substrate, wherein the substrate itself may include a plurality of thermochromic regions that may be activatable at distinct thermal thresholds, pulse widths, heat-transfer pressure levels, or wavelength-selective infrared absorption profiles. In some embodiments, the system may include the communication device, processing device, printing device, drug database interface, and IV pump integration mechanisms, while also implementing the novel multi-color thermal substrate and multi-temperature activation systems.

In some embodiments, the present disclosure may therefore introduce a synergistic technical framework in which the label-generation logic may dynamically determine the therapeutic, temporal, and operational requirements of an IV infusion line and may map those requirements onto a thermally activatable color scheme that may be printed in a plurality of colors on a single label without using any pigments, ribbons, or ink cartridges. In some embodiments, the system may substantially improve computer-implemented labeling workflows, multi-color thermal printing technology, thermochromic substrate chemistry, drug metadata processing, and clinical safety systems.

In some embodiments, the disclosed system may provide an advanced thermochromic label substrate comprising multiple color-producing layers or regions, each of which may activate at a distinct thermal threshold, wherein the processing device may automatically determine which thermochromic color region must be activated at each pixel location based on drug class, tubing-change urgency, day-of-week indicators, infection-prevention rules, and institution-defined color schemes. The technical problem addressed is that existing thermal printing systems typically activate only a single color (black) because conventional thermal papers contain only one thermochromic layer, preventing the automated generation of multi-color alerts such as multi-hour tubing-change time windows, drug-class-specific hazard colors, or day-of-week color coding. Hospitals therefore maintain large inventories of pre-printed labels, which are inflexible and incompatible with dynamically calculated content such as tubing-change deadlines or drug-specific warnings.

In some embodiments, the substrate of the disclosed system may include a low-temperature activatable yellow layer, a medium-temperature activatable blue layer, and a higher-temperature activatable black layer, or any combination thereof, with or without an orange layer that may activate at still higher temperatures. In some embodiments, the printhead may produce controlled thermal pulses such that only the targeted thermochromic layer activates without disturbing deeper or adjacent layers. In some embodiments, the substrate may be constructed as stacked layers separated by micro-insulating films; alternatively, the substrate may be formed as spatially segmented regions each individually coated with thermochromic dye chemistries tuned to particular activation thresholds. In some embodiments, the specific technology improved by the given feature is thermal printing technology, including the physics of thermal printhead actuation, heat-transfer patterns, and color selection on multi-layer thermal media.

In some embodiments, the disclosed system may allow the processing device to automatically calculate tubing-change deadlines for any drug identified via barcode, RFID, or manual entry. The technical problem addressed is that existing systems rely on handwritten calculations prone to human error and may not automatically generate color-coded urgency indicators.

In some embodiments, the combined system may map the time-remaining window to a thermochromic color pattern printed directly onto the multi-colored substrate. For example, the system may print a yellow band when more than four hours remain, an orange band when between one and four hours remain, and a red-equivalent thermochromic color when one hour or less remains. Because different colors may be generated via selective temperature pulses, the printer may perform a multi-pass or single-pass thermal activation sequence to output a fully multi-color tubing alert without pre-printed stock.

In some embodiments, the specific technology improved by the given feature is clinical workflow automation and drug-metadata-driven color coding, implemented through integration of thermochromic color control with IV-drug metadata processing.

In some embodiments, the printhead may include individually controllable micro-heating elements configured to deliver custom temperature pulses to each pixel row and column, allowing adjacent pixels to exhibit entirely different colors. The technical problem addressed is that conventional thermal printers may not generate high-resolution multi-color images because heat diffuses laterally across the substrate, causing color bleed and restricting adjacent pixels to similar temperature outputs.

In some embodiments, the system may mitigate the given problem by:
Engineering micro-insulating channels or micro-etched lattices beneath the thermochromic coating to reduce thermal crosstalk.
Introducing anisotropic thermal-conductivity pathways that direct heat vertically while limiting lateral spread.
Applying pulse-width modulation such that shallow thermochromic layers activate without causing heat penetration into deeper layers.

In some embodiments, the specific technology improved is pixel-level multi-color thermal imaging, enabling multi-color barcodes, multi-color machine-readable embellishments, and micro-segmented alert patterns.

In some embodiments, the disclosed system may integrate the biometric sensor and authentication logic with multi-color substrate capabilities, enabling thermochromic color patterns that may represent whether a label was generated by an authenticated clinician. The technical problem addressed is that critical labels associated with narcotics or high-risk drugs may need explicit confirmation that they were created by authorized personnel. Existing systems rely on printed operator names, which may be spoofed or mis-entered. The disclosed system may embed a color-coded authentication indicator that may not be altered or forged after thermal activation.

In some embodiments, the processing device may generate a color pattern (e.g., blue diagonal bars) only after the biometric signature is validated. In some embodiments, the given color indicator may not activate without a specific combination of heat and pressure, making it resistant to tampering.

In some embodiments, the specific technology improved is secure biometric-validated printing, using multi-chromic thermal activation as a hardware-enforced authentication signal.

In some embodiments, when the communication device may not find a scanned drug in the printer's internal database, the system may automatically query external hospital databases, cloud-hosted drug information sources, or local fallback menus. The technical problem addressed is that compounded drugs, pediatric formulations, and NICU-specific preparations often lack standard barcodes and therefore may not be assigned correct tubing-change times or clinical hazard colors.

In some embodiments, once drug metadata is retrieved or selected, the system may automatically map it to a color-coded thermochromic pattern indicating the drug category, stability, infection risk level, or required storage-temperature range. For example, a thermochromic patch that turns purple at 40° C. may signify that a refrigerated drug has been exposed to unsafe conditions.

In some embodiments, the specific technology improved is drug-database integration with thermochromic output, merging pharmacological metadata with hardware-driven color selection.

In some embodiments, the IV infusion pump may transmit metadata to the printer (e.g., drug identity, start time, infusion rate), and the processing device may automatically generate color-coded thermochromic alerts. The technical problem addressed is that infusion pumps and label printers traditionally operate independently, causing mismatches between pump settings, tubing-change timelines, and printed label content.

In some embodiments, label-generation logic may automatically adjust the thermochromic activation sequence to reflect pump-specific metadata. For example, if the pump is set to deliver a vesicant drug, the system may print an irreversible red alert box using a deep-activation thermochromic layer; if the pump indicates a secondary (piggyback) line configuration, the color may shift according to secondary-line infection-risk rules.

In some embodiments, the specific technology improved is automated IV pump- printer synchronization, enabling self-updating multi-color safety labels.

In some embodiments, the label substrate may include reversible thermochromic zones that change color based on ambient temperature or elapsed time, and irreversible zones that permanently activate upon exceeding a threshold temperature. The technical problem addressed is that clinicians currently lack a self-indicating label that visually differentiates between permanent information (drug name, tubing-change deadline) and dynamic information (infusion paused, refrigeration breach, elapsed-time indicators).

In some embodiments, reversible dyes may gradually fade to indicate elapsed time since label creation, creating a time-temperature exposure graph directly on the label. In some embodiments, irreversible dyes may permanently activate when the substrate receives a high-temperature pulse from the printer during initial printing, ensuring that core drug information may not be altered later.

In some embodiments, the specific technology improved is hybrid reversible/irreversible thermochromic labeling, enabling built-in monitoring of environmental conditions and temporal exposure.

In some embodiments, the processing device may execute a machine-learning model trained on the thermal response characteristics of the multi-layer substrate. The technical problem addressed is that manual calibration of heat pulses fails to compensate for real-world variations such as humidity, paper thickness, thermochromic dye aging, and printhead wear.

In some embodiments, the model may dynamically adjust temperature, pulse width, pressure, or pre-heat patterns in real time. In some embodiments, a neural network may predict the optimal pulse sequence for each pixel to achieve the intended color without bleed.

In some embodiments, the improved technology is adaptive thermal-printing control, improving multi-color accuracy and long-term printer reliability.

In some embodiments, the system may include photothermal absorbers within the substrate that may selectively absorb laser or LED illumination, allowing optical pre-heating of selected zones before thermal printhead contact.

The technical problem addressed is that high-temperature activation requires substantial electrical energy and may degrade printheads prematurely.

In some embodiments, the printer may project wavelength-specific light onto particular zones containing photothermal nanoparticles that absorb the radiation and convert to localized heat, thereby reducing the required printhead temperature.

In some embodiments, the technology improved is energy-efficient multi-color thermal printing using hybrid photothermal-printhead activation.

In some embodiments, the system may generate continuous gradient coloration rather than discrete colors by applying thermal gradients across a region. The technical problem addressed is that abrupt color transitions may reduce readability or obscure nuanced clinical timing windows.

In some embodiments, the printer may sweep the printhead or use modulated heating sequences to create a smooth transition from yellow to orange to black, representing increasing urgency of an upcoming tubing-change event.

In some embodiments, the improved technology is continuous-tone thermochromic imaging, enabling gradient-based clinical indicators.

In some embodiments, the substrate may include printed conductive traces that channel heat flux from the printhead into specific sub regions. The technical problem addressed is that conventional thermal media distributes heat uniformly, making impossible to create complex shapes or symbols without precise direct heating.

In some embodiments, heat-steering traces may cause heat to flow laterally or vertically into specific symbol patterns (e.g., hazard triangles) even when the printhead delivers a uniform heat pulse.

In some embodiments, the technology improved is substrate-level heat-flow engineering, enabling low-energy creation of complex multi-color shapes.

In some embodiments, the substrate may include micro-ridges, lattice structures, or embossed micro-channels that physically modulate heat transfer. The technical problem addressed is lateral heat spread, which causes color bleeding and reduces multi-color resolution.

In some embodiments, micro-patterns may prevent adjacent thermochromic regions from activating inadvertently, allowing high-definition multi-color symbols even at very small scales.

In some embodiments, the improved technology is micro-engineered thermal isolation, which increases the achievable resolution of multi-color thermal printing.

In some embodiments, the present disclosure may represent a novel and non-obvious integration of: (1) an intelligent IV-infusion label-generation system with advanced data-processing capabilities and drug-metadata logic; and (2) a multi-layer, temperature-selective thermochromic substrate and advanced thermal-activation system enabling multi-color output from a standard thermal printer.

Further, in some embodiments, the given integrated system may enhance the underlying technologies of thermal printing, drug-safety automation, IV workflow management, high-precision thermochromic chemistry, thermal imaging, and clinical informatics, thereby solving longstanding technical problems associated with single-color thermal printers, manual labeling, pre-printed color inventory, tubing-change miscalculations, and unsafe clinical labeling practices.

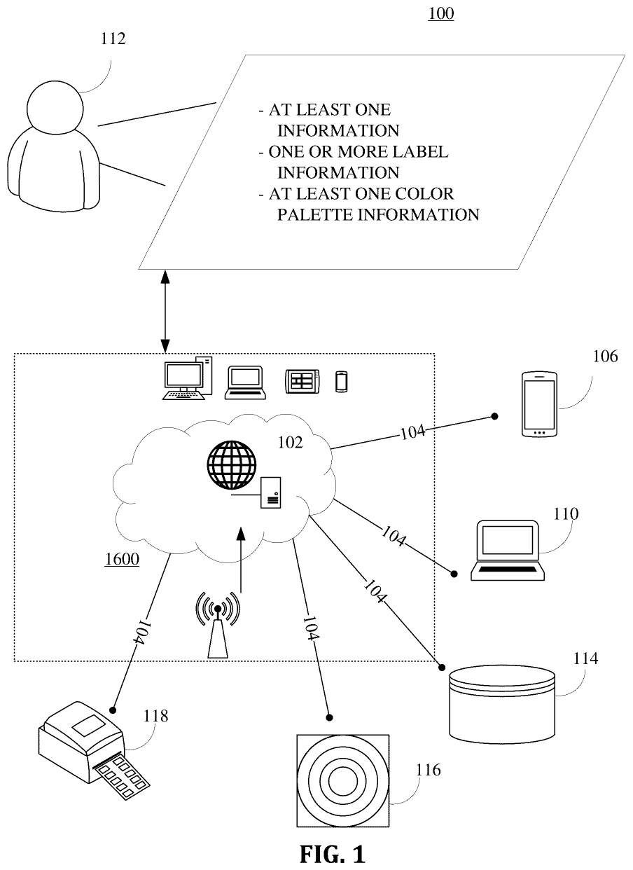

FIG. 1 is an illustration of an online platform 100 consistent with various embodiments of the present disclosure. By way of non-limiting example, the online platform 100 to facilitate creating labels for labeling intravenous infusion lines may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, sensors 116, actuators (not shown) and a device 118 (such as a device 1100) over a communication network 104, such as, but not limited to, the Internet. Further, users of the online platform 100 may include relevant parties such as, but not limited to, end-users, and administrators. Accordingly, in some instances, electronic devices operated by the one or more relevant parties may be in communication with the online platform 100.

A user 112, such as the one or more relevant parties, may access the online platform 100 through a web-based software application or browser. The web-based software application may be embodied as, for example, but not be limited to, a website, a web application, a desktop application, and a mobile application compatible with a computing device 1600.

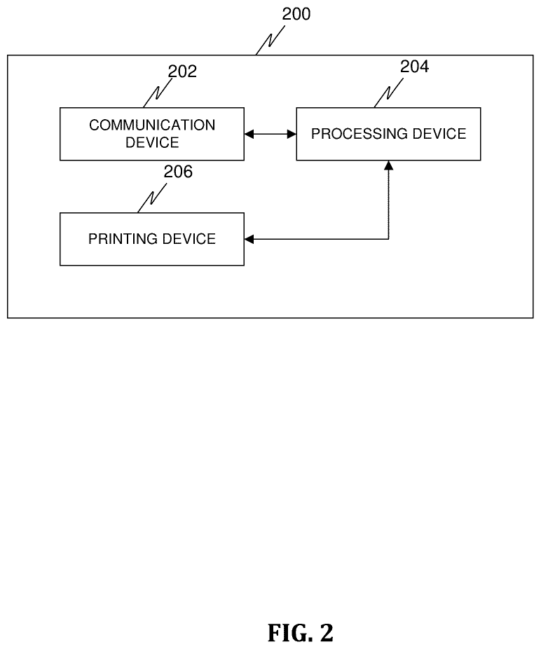

FIG. 2 is a block diagram of a system 200 for creating labels for labeling intravenous infusion lines, in accordance with some embodiments. Accordingly, the system 200 may include a communication device 202, a processing device 204, and a printing device 206.

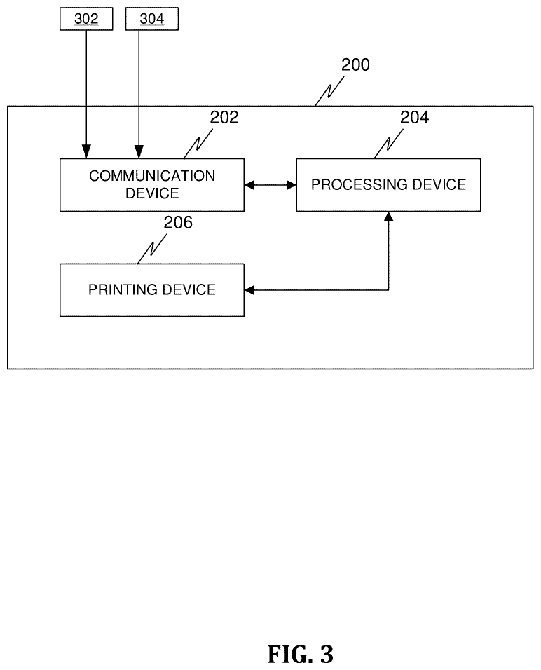

Further, the communication device 202 may be configured for receiving at least one information associated with at least one intravenous infusion line from one or more input devices 302-304 (as shown in FIG. 3). Further, the one or more input devices 302-304 may include a scanner, a RFID reader, a computing device, an IV infusion pump, etc. Further, the one or more input devices 302-304 may be configured for scanning at least one visual representation of the at least one information present on at least one physical object. Further, the at least one visual representation may include a QR code, a barcode, etc. Further, the at least one physical object may include a prescription, a medicine container, a tag, a user's identification, a medicine's identification, a doctor's identification, etc. Further, the at least one information may include user information, medicine information, doctor information, etc. Further, the computing device may include a smartphone, a laptop, a desktop, a smartwatch, etc.

Further, the processing device 204 may be communicatively coupled with the communication device 202. Further, the processing device 204 may be configured for analyzing the at least one information. Further, the processing device 204 may be configured for determining one or more label content of one or more labels associated with the at least one intravenous infusion line based on the analyzing. Further, the one or more label content may include user's information, medicine information, time and date of administration, required change time for the infusion tubing, drug-specific warnings, and location of the infusion's administration on a patient, etc. Further, the processing device 204 may be configured for determining at least one label content format of the one or more label content based on the determining of the one or more label content. Further, the at least one label content format may include a color code for the one or more label content. Further, the color code may include at least seven different colors for seven days of the week. Further, the processing device 204 may be configured for generating one or more label information for the one or more labels based on the one or more label content and the at least one label content format.

Further, the printing device 206 may be communicatively coupled with the processing device 204. Further, the printing device 206 may be configured for printing the one or more label information on one or more label sheets based on the generating of the one or more label information. Further, the printing of the one or more label information may create the one or more labels for the at least one intravenous infusion line. Further, the printing may include thermal printing of the one or more label information on the one or more label sheets. Further, the one or more label information may be thermally printed on the one or more label sheets. Further, the one or more label information may include the one or more label content in the at least one label content format.

In some embodiments, the one or more labels may include adhesive.

In some embodiments, the printing device 206 may include a thermal printer.

In some embodiments, the one or more input devices 302-304 may include one or more of an optical scanner, an RFID reader, and a computing device.

In some embodiments, the optical scanner may be configured for scanning one or more visual representations of the one or more information presents on one or more physical objects.

In some embodiments, the one or more label content formats may include a color code for the one or more label content. Further, the color code may include at least seven different colors corresponding to seven days of a calendar week.

In some embodiments, the one or more labels may include a primary label and one or more secondary labels. Further, a primary label content of the primary label may include user's information, medicine information, time and date of administration, a required tubing change time for intravenous infusion, a drug-specific warning, and location of the infusion′ may s administration. Further, the one or more secondary labels may be configured for tagging the one or more intravenous infusion lines in one or more places of the one or more intravenous infusion lines. Further, the one or more secondary labels may include drug name and administration location information.

In some embodiments, the communication device 202 may be configured for receiving drug information associated with a drug from one or more drug databases. Further, the processing device 204 may be configured for calculating a required tubing change time for intravenous infusion of the drug based on the drug information.

FIG. 3 illustrates a block diagram of the system 200 for creating a label for labeling an intravenous infusion line, in accordance with some embodiments.

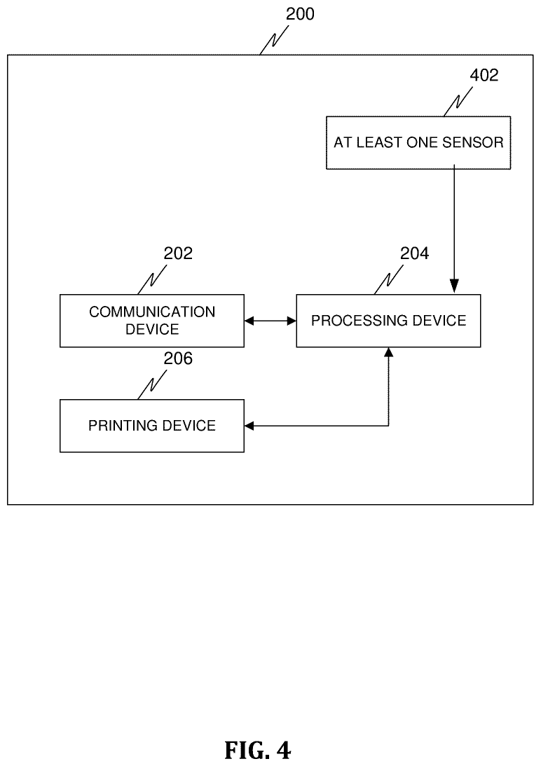

FIG. 4 illustrates a block diagram of the system 200 for creating a label for labeling the intravenous infusion line, in accordance with some embodiments.

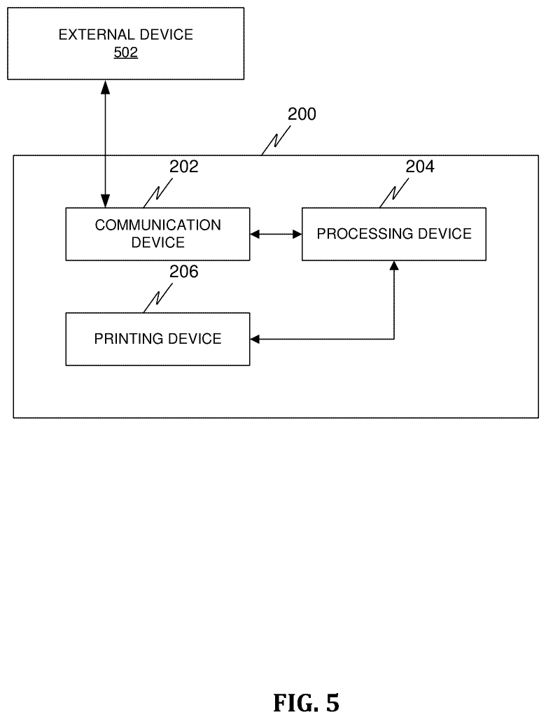

In some embodiments, the system 200 may include one or more sensors 402 (as shown in FIG. 4) communicatively coupled to the processing device 204. Further, the one or more sensors 402 may be configured for generating tracking data associated with a labeling activity. Further, the communication device 202 may be configured for transmitting each of the tracking data and the one or more information to an external device 502 (as shown in FIG. 5) for auditing. Further, the one or more information may include a label administrator's information.

FIG. 5 illustrates a block diagram of the system 200 for creating a label for labeling the intravenous infusion line, in accordance with some embodiments.

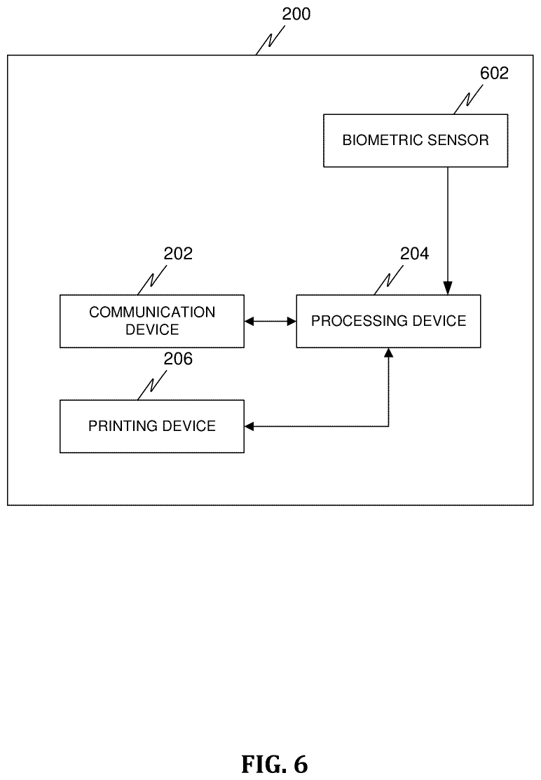

FIG. 6 illustrates a block diagram of the system 200 for creating a label for labeling the intravenous infusion line, in accordance with some embodiments.

In some embodiments, the system 200 may include a biometric sensor 602 (as shown in FIG. 6) communicatively coupled to the processing device 204. Further, the biometric sensor 602 may be configured for generating biometric data of a label administrator. Further, the processing device 204 may be configured for performing authentication of the label administrator based on the biometric data.

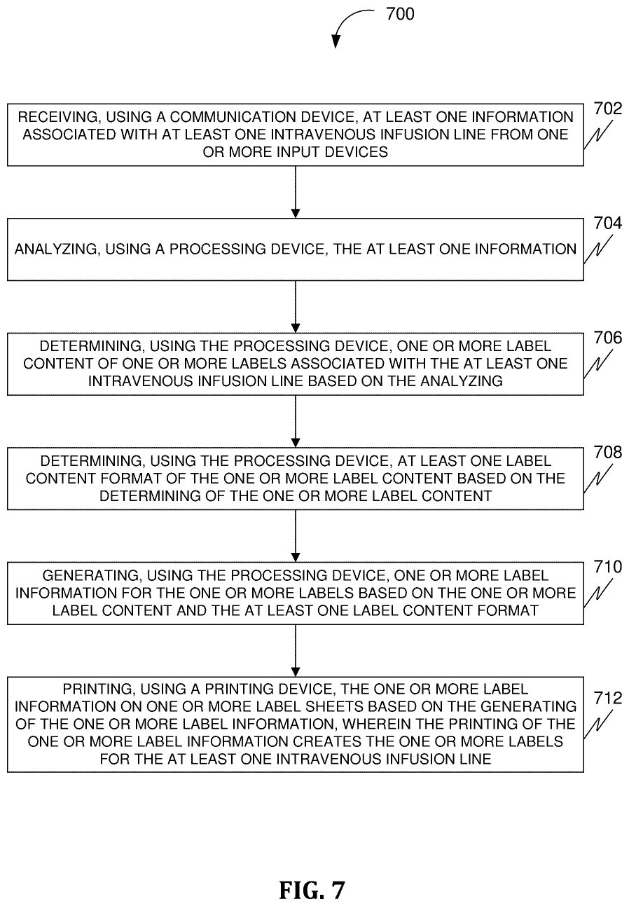

FIG. 7 illustrates a flowchart of a method 700 for creating a label for labeling an intravenous infusion line, in accordance with some embodiments. Accordingly, the method 700 may include a step 702 of receiving, using a communication device (such as the communication device 202), at least one information associated with at least one intravenous infusion line from one or more input devices (such as the one or more input devices 302-304).

Further, the method 700 may include a step 704 of analyzing, using a processing device (such as the processing device 204), the at least one information.

Further, the method 700 may include a step 706 of determining, using the processing device, one or more label content of one or more labels associated with the at least one intravenous infusion line based on the analyzing.

Further, the method 700 may include a step 708 of determining, using the processing device, at least one label content format of the one or more label content based on the determining of the one or more label content.

Further, the method 700 may include a step 710 of generating, using the processing device, one or more label information for the one or more labels based on the one or more label content and the at least one label content format.

Further, the method 700 may include a step 712 of printing, using a printing device (such as the printing device 206), the one or more label information on one or more label sheets based on the generating of the one or more label information, wherein the printing of the one or more label information creates the one or more labels for the at least one intravenous infusion line.

In some embodiments, the one or more labels may include adhesive.

In some embodiments, the printing device may include a thermal printer.

In some embodiments, the one or more input devices may include one or more of an optical scanner, an RFID reader, and a computing device.

In some embodiments, the method 700 may include scanning, using the optical scanner, one or more visual representations of the one or more information presents on one or more physical objects.

In some embodiments, the one or more label content formats may include a color code for the one or more label content. Further, the color code may include at least seven different colors corresponding to seven days of a calendar week.

In some embodiments, the one or more labels may include a primary label and one or more secondary labels. Further, a primary label content of the primary label may include user's information, medicine information, time and date of administration, a required tubing change time for intravenous infusion, a drug-specific warning, and location of the infusion' may s administration. Further, the one or more secondary labels may be configured for tagging the one or more intravenous infusion lines in one or more places of the one or more intravenous infusion lines. Further, the one or more secondary labels may include drug name and administration location information.

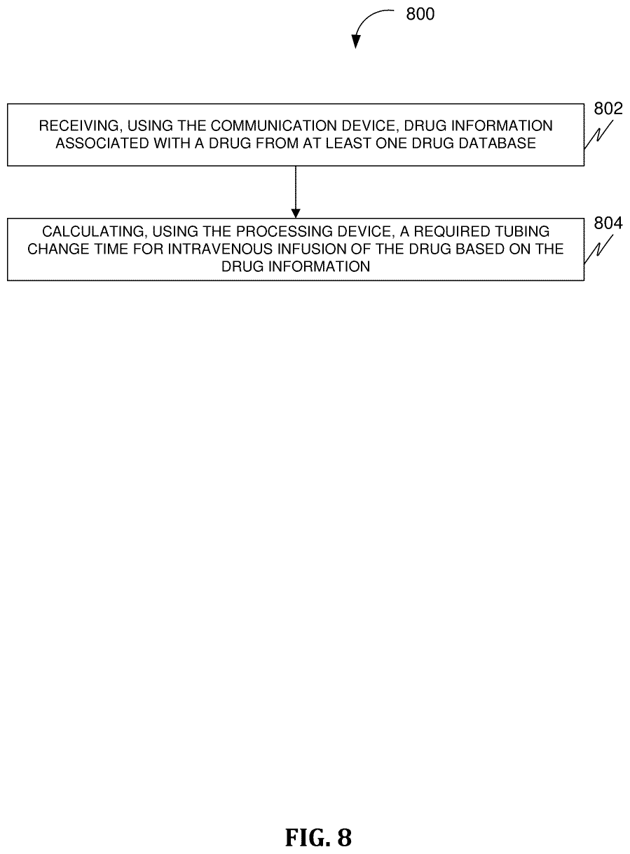

FIG. 8 illustrates a flowchart of a method 800 for creating a label for labeling an intravenous infusion line, in accordance with some embodiments.

Further, the method 800 may include a step 802 of receiving, using the communication device, drug information associated with a drug from at least one drug database.

Further, the method 800 may include a step 804 of calculating, using the processing device, a required tubing change time for intravenous infusion of the drug based on the drug information.

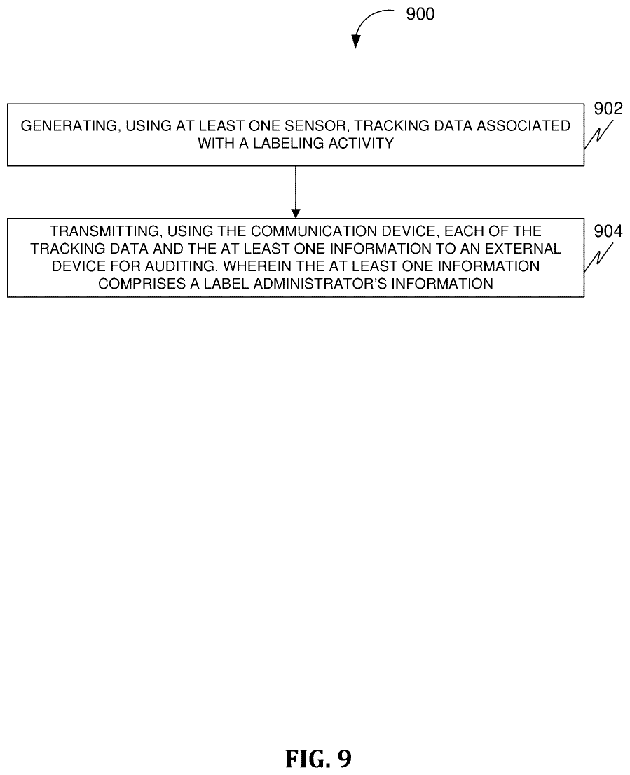

FIG. 9 illustrates a flowchart of a method 900 for creating a label for labeling an intravenous infusion line, in accordance with some embodiments.

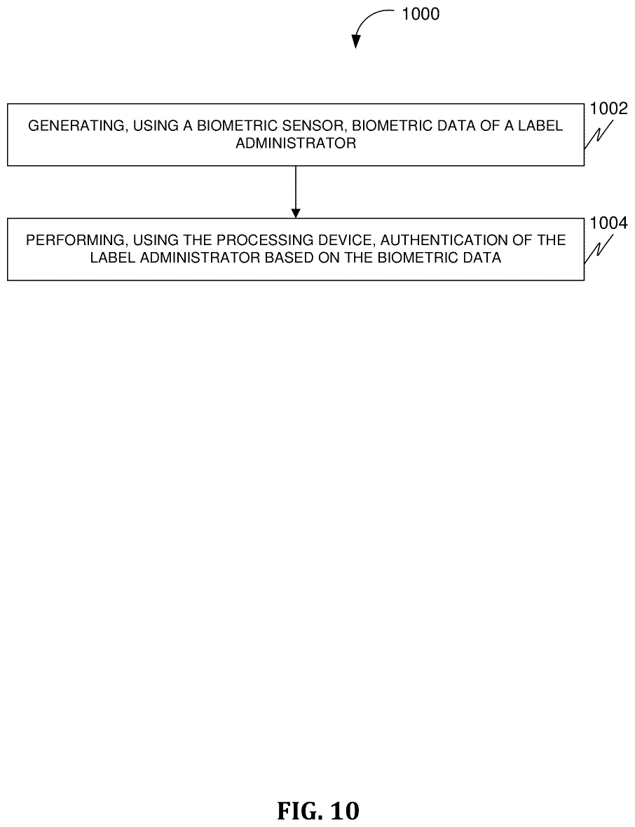

Further, the method 900 may include a step 902 of generating, using at least one sensor (such as the one or more sensors 402), tracking data associated with a labeling activity. Further, the method 900 may include a step 904 of transmitting, using the communication device, each of the tracking data and the at least one information to an external device (such as the external device 502) for auditing, wherein the at least one information comprises a label administrator's information FIG. 10 illustrates a flowchart of a method 1000 for creating a label for labeling an intravenous infusion line, in accordance with some embodiments. Further, the method 1000 may include a step 1002 of generating, using a biometric sensor (such as the biometric sensor 602), biometric data of a label administrator. Further, the method 1000 may include a step 1004 of performing, using the processing device, authentication of the label administrator based on the biometric data.

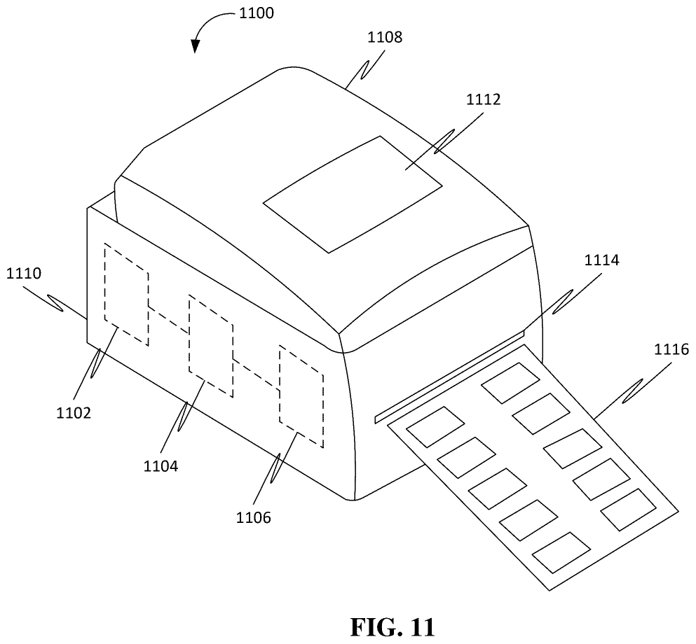

FIG. 11 is a top perspective view of a device 1100 for creating labels for labeling intravenous infusion lines, in accordance with some embodiments. Further, the device 1100 may include a communication device 1102, a processing device 1104, and a printing device 1106.

Further, the device 1100 may include a top 1108 pivotally connected to a base 1110. Further, the device 1100 may be configured for carrying one or more label sheets 1116. Further, the top 1108 may be pivotable between a closed position and at least one open position. Further, the device 1100 may include a cavity formed by the base 1110 for receiving the one or more label sheets 1116. Further, the cavity may be accessible to a user for disposing the one or more label sheets 1116 in the cavity in the at least one open position.

Further, the printing device 1106 may be enclosed within the base 1110. Further, the printing device 1106 may be configured for printing one or more label information on the one or more label sheets 1116 based on feeding of the one or more label sheets 1116. Further, the one or more label sheets 1116 may exit through an opening 1114 of the apparatus after the printing. Further, the one or more label sheets 1116 exited from the opening 1114 may be accessible to the user.

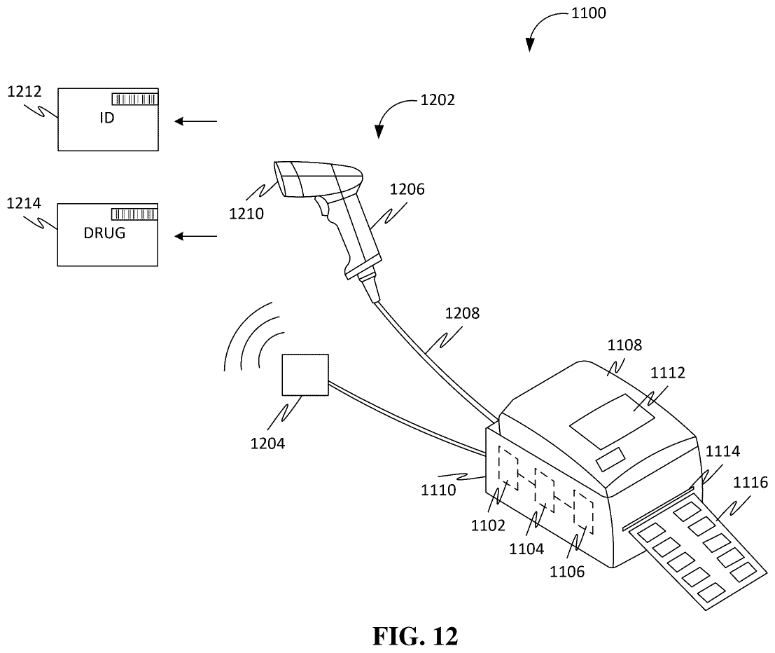

Further, the communication device 1102 may be configured for receiving at least one information associated with at least one intravenous infusion line from one or more input devices 1202-1204, as shown in FIG. 12. Further, the one or more input devices 1202-1204 may include a scanner 1202, an RFID reader 1204, etc. Further, the one or more input devices 1202-1204 may be configured for scanning at least one visual representation of the at least one information present on at least one physical object. Further, the at least one visual representation may include a QR code, a barcode, etc. Further, the at least one physical object may include a prescription, a medicine container, a tag, a doctor's identification, a user's identification 1212 (as shown in FIG. 12), a patient's identification, a medicine's identification 1214 (as shown in FIG. 12), etc. Further, the at least one information may include user information, medicine information, doctor information, etc.

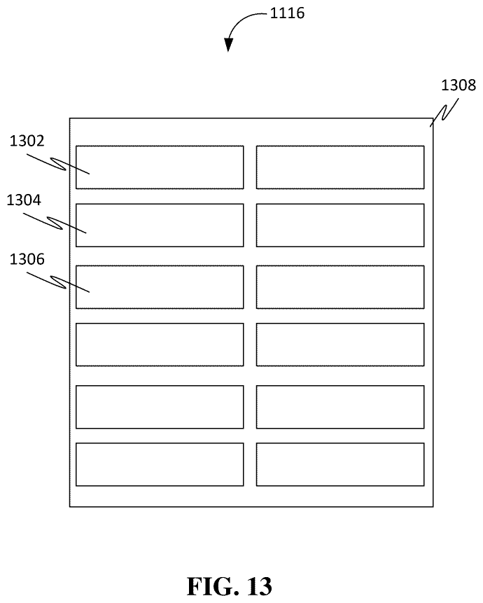

Further, the processing device 1104 may be communicatively coupled with the communication device 1102 and the printing device 1106. Further, the processing device 1104 may be configured for analyzing the at least one information. Further, the processing device 1104 may be configured for determining one or more label content of one or more labels 1302-1306, as shown in FIG. 13, associated with the at least one intravenous infusion line based on the analyzing. Further, the processing device 1104 may be configured for determining at least one label content format of the one or more label content based on the determining of the one or more label content. Further, the processing device 1104 may be configured for generating the one or more label information for the one or more labels 1302-1306 based on the one or more label content and the at least one label content format. Further, the printing device 1106 may be configured for printing the one or more label information on the one or more label sheets 1116 based on the generating of the one or more label information. Further, the printing of the one or more label information may create the one or more labels 1302-1306 for the at least one intravenous infusion line. Further, the printing of the one or more label information greatly reduces the time and effort to create the one or more labels 1302-1306 for the at least one intravenous infusion line and potentially eliminates human error. Further, the printing may include thermal printing of the one or more label information on the one or more label sheets 1116. Further, the one or more label information may be thermally printed on the one or more label sheets 1116. Further, the one or more label information may include the one or more label content in the at least one label content format.

Further, in some embodiments, the device 1100 may be provided with a display 1112 carried on the top 1108 and in viewing access to the user. Further, in some embodiments, the display 1112 displays information related to creating of the one or more labels 1302-1306 e.g., staff information, medicine information, date, time, and so forth, in addition to displaying prompts (e.g., “Scan Drug NDC”) and information related to device's setup and device's settings.

Further, in some embodiments, the display 1112 may be manipulated by the user to manually input information, for example, with the use of a touch screen of the display 1112. Further, the display 1112 disposed on the top 1108 may be configured to read fingerprints of the user for security verification of the user prior to use.

Further, in some embodiments, the device 1100 may include one or more ports that allow communication with a computer for preprogramming.

Further, in some embodiments, the device 1100 may include a power cord (not shown) that provides electrical energy to the device 1100 via an electrical outlet.

Further, in some embodiments, the one or more input devices 1202-1204 may be communicatively coupled with the communication device 1102 using at least one of a wired communication channel and a wireless communication channel. Further, the one or more input devices 1202-1204 may include the scanner 1202. Further, the device 1100 associated with the scanner 1202 conductively connected with the device 1100 via a cord 1208, or associated with a mobile or stationary computer which includes a first scanner. Further, the scanner 1202 includes a handle 1206 integrally attached to a reader 1210. During use, the user may hold the handle 1206 and scan using the reader 1210, for example, a barcode associated with the user's identification 1212 and a barcode associated with the medicine's identification 1214 being used, as indicated by a medicine's box. Further, medicine bottles include barcodes adhered to the medicine bottle, which provide important medical information. Thus, the medicine's box represents a barcode directly from the medicine bottle.

FIG. 12 is a top perspective view of the device 1100 with the one or more input devices 1202-1204, in accordance with some embodiments.

FIG. 13 is a front view of the one or more label sheets 1116, in accordance with some embodiments. Further, the one or more label sheets 1116 may include the one or more labels 1302-1306 adhesively bonded to one or more sheets 1308. Further, the one or more labels 1302-1306 may include the one or more label information printed on the one or more labels 1302-1306. Further, the one or more labels 1302-1306 are removable from the one or more sheets 1308. Further, the one or more labels 1302-1306 adhesively adhere to the at least one intravenous infusion line. Further, in some embodiments, the one or more labels 1302-1306 do not include the one or more label information printed on the one or more labels 1302-1306. Further, the one or more labels 1302-1306 may include one or more prompts of the one or more label information printed on the one or more labels 1302-1306.

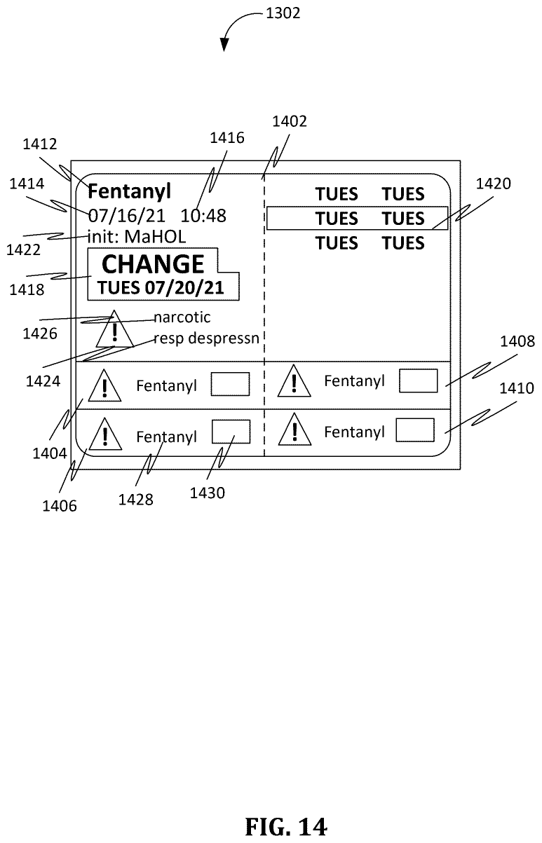

FIG. 14 is a front view of a label 1302 of the one or more labels 1302-1306, in accordance with some embodiments. Further, the label 1302 may include a primary label 1402 and one or more secondary labels 1404-1410. Further, the primary label 1402 may include one or more key information related to an intravenous infusion provided to the user using the at least one intravenous infusion line. Further, the one or more secondary labels 1404-1410 may be intended for tagging the at least one intravenous infusion line in one or more places of the at least one intravenous infusion line. Further, the one or more secondary labels 1404-1410 may be printed concurrently with the primary label 1402. Further, the primary label 1402 may include the drug name in a section 1412 of the primary label 1402, date of administration in a section 1414 of the primary label 1402, time of administration in a section 1416 of the primary label 1402, required tubing change time printed in a section 1418 of the primary label 1402, color-coded required tubing change time alert in a section 1420 of the primary label 1402, user (administrator) information in a section 1422 of the primary label 1402, drug warning in a section 1424 of the primary label 1402, and drug type information in a section 1426 of the primary label 1402. Further, a secondary label 1406 of the one or more secondary labels 1404-1410 may include drug name in a section 1428 of the secondary label 1406 and administration location information in a section 1430 of the secondary label 1406.

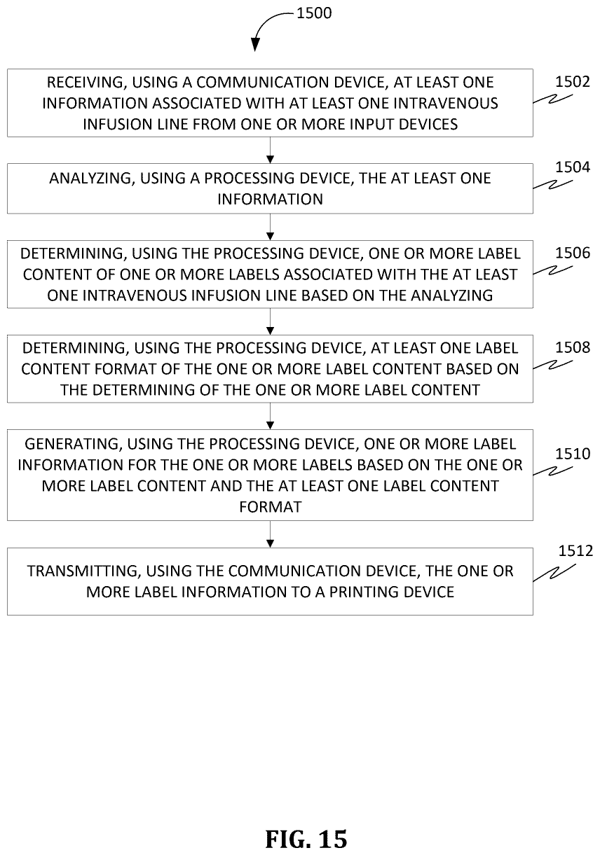

FIG. 15 is a flowchart of a method 1500 for creating labels for labeling intravenous infusion lines, in accordance with some embodiments. Accordingly, at 1502, the method 1500 may include a step of receiving, using a communication device (such as the communication device 202), at least one information associated with at least one intravenous infusion line from one or more input devices.

Further, at 1504, the method 1500 may include a step of analyzing, using a processing device (such as the processing device 204), the at least one information.

Further, at 1506, the method 1500 may include a step of determining, using the processing device, one or more label content of one or more labels associated with the at least one intravenous infusion line based on the analyzing.

Further, at 1508, the method 1500 may include a step of determining, using the processing device, at least one label content format of the one or more label content based on the determining of the one or more label content.

Further, at 1510, the method 1500 may include a step of generating, using the processing device, one or more label information for the one or more labels based on the one or more label content and the at least one label content format.

Further, at 1512, the method 1500 may include a step of transmitting, using the communication device, the one or more label information to a printing device (such as the printing device 206). Further, the printing device may be configured for printing the one or more label information on the one or more label sheets based on the generating of the one or more label information. Further, the printing of the one or more label information may create the one or more labels for the at least one intravenous infusion line. Further, the printing may include thermal printing of the one or more label information on the one or more label sheets. Further, the one or more label information may be thermally printed on the one or more label sheets. Further, the one or more label information may include the one or more label content in the at least one label content format.

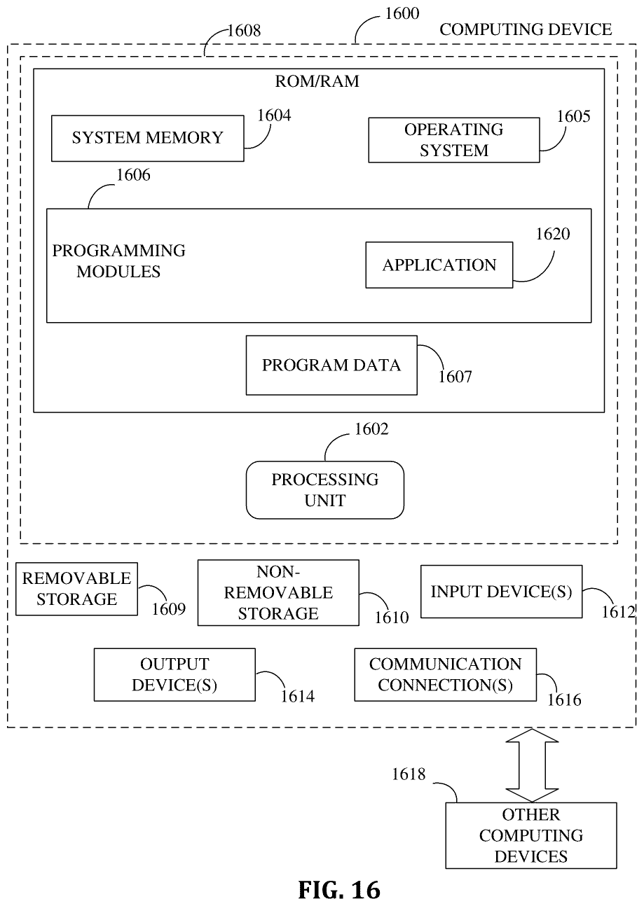

With reference to FIG. 16, a system consistent with an embodiment of the disclosure may include a computing device or cloud service, such as computing device 1600. In a basic configuration, computing device 1600 may include at least one processing unit 1602 and a system memory 1604. Depending on the configuration and type of computing device, system memory 1604 may comprise, but is not limited to, volatile (e.g., random-access memory (RAM)), non-volatile (e.g., read-only memory (ROM)), flash memory, or any combination. System memory 1604 may include operating system 1605, one or more programming modules 1606, and may include a program data 1607. Operating system 1605, for example, may be suitable for controlling computing device 1600's operation. In one embodiment, programming modules 1606 may include image-processing module, machine learning module and/or image classifying module. Furthermore, embodiments of the disclosure may be practiced in conjunction with a graphics library, other operating systems, or any other application program and is not limited to any particular application or system. This basic configuration is illustrated in FIG. 16 by those components within a dashed line 1608.

Computing device 1600 may have additional features or functionality. For example, computing device 1600 may also include additional data storage devices (removable and/or non-removable) such as, for example, magnetic disks, optical disks, or tape. Such additional storage is illustrated in FIG. 16 by a removable storage 1609 and a non-removable storage 1610. Computer storage media may include volatile and nonvolatile, removable and non-removable media implemented in any method or technology for storage of information, such as computer-readable instructions, data structures, program modules, or other data. System memory 1604, removable storage 1609, and non-removable storage 1610 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1600. Any such computer storage media may be part of device 1600. Computing device 1600 may also have input device(s) 1612 such as a keyboard, a mouse, a pen, a sound input device, a touch input device, a location sensor, a camera, a biometric sensor, etc. Output device(s) 1614 such as a display, speakers, a printer, etc. may also be included. The aforementioned devices are examples and others may be used.

Computing device 1600 may also contain a communication connection 1616 that may allow device 1600 to communicate with other computing devices 1618, such as over a network in a distributed computing environment, for example, an intranet or the Internet. Communication connection 1616 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 1604, including operating system 1605. While executing on processing unit 1602, programming modules 1606 (e.g., application 1620 such as a media player) may perform processes including, for example, one or more stages of methods, algorithms, systems, applications, servers, databases as described above. The aforementioned process is an example, and processing unit 1602 may perform other processes. Other programming modules that may be used in accordance with embodiments of the present disclosure may include sound encoding/decoding applications, machine learning application, acoustic classifiers, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

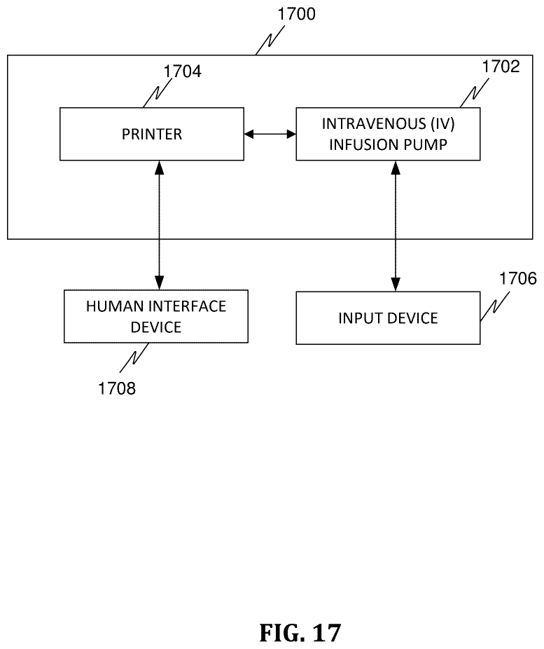

FIG. 17 is a block diagram of a system 1700 for creating a label for labeling an intravenous infusion line, in accordance with some embodiments. Accordingly, the system 1700 may include a printer 1704 and an intravenous (IV) infusion pump 1702.

Further, the IV infusion pump 1702 may be configured for delivering at least one drug to at least one user using at least one intravenous infusion line. Further, the IV infusion pump 1702 may be programmable. Further, the IV infusion pump 1702 may be configured for receiving one or more information through at least one input device 1706 associated with the IV infusion pump 1702 for programming the IV infusion pump 1702. Further, the at least one input device 1706 may include one or more buttons on the IV infusion pump 1702. Further, the delivering of the at least one drug to the at least one user using the at least one intravenous line may be based on the programming. Further, the one or more information may include metadata such as a nurse's identification of a nurse, a nurse's name of the nurse, a drug's information of the at least one drug, etc. Further, the IV infusion pump 1702 may be configured for transmitting the metadata to the printer 1704. Further, the IV infusion pump 1702 may be comprised of a communication device.

Further, the printer 1704 may be configured for receiving the metadata from the IV infusion pump 1702. Further, the printer 1704 may be configured for processing the metadata. Further, the printer 1704 may be configured for determining one or more label content of one or more labels associated with the at least one intravenous infusion line based on the processing. Further, the printer 1704 may be configured for determining one or more label content formats of the one or more label content based on the determining of the one or more label content. Further, the printer 1704 may be configured for generating one or more label information for the one or more labels based on the one or more label content and the one or more label content formats. Further, the printer 1704 may be configured for printing the one or more label information on one or more label sheets based on the generating of the one or more label information. Further, the printing of the one or more label information creates the one or more labels for the at least one intravenous infusion line. Further, the printer 1704 may be comprised of a processing device and a printing device.

Further, in some embodiments, the at least one label content format may include a color code for the one or more label content. Further, the color code may include at least three different colors. Further, two of the three different colors correspond to two specific days of a week on which the at least one intravenous infusion line may be changed by a hospital as per a routine of the hospital. Further, the two specific days of the week may be Mondays and Thursdays. Further, a first color and a second color of the three colors may represent Mondays and Thursdays. Further, a third color of the three colors represents a day that may be a change date of a drug such as Propofol which needs to be changed much sooner than either Monday or Thursday.

Further, in some embodiments, the at least one label content format may include a color code for the one or more label content. Further, the one or more label content may include a drug's name, a medicine name, etc. Further, the color code for the drug's name, the medicine name, etc. may represent a type of drug and/or medicine such as narcotics.

Further, in some embodiments, the one or more labels may include one or more thermal labels. Further, the one or more thermal labels may have different colors instead of black under the white finish.

Further, in some embodiments, the printer 1704 may be configured for determining an absence of a drug of the at least one drug in a database of the printer 1704 based on the processing. Further, the printer 1704 may be configured for generating a prompt for the nurse to select the drug and/or a number of hours to expire for the one or more labels. Further, the printer 1704 may be configured for displaying the prompt using a human interface device 1708 associated with the printer 1704. Further, the printer 1704 may be configured for receiving a selection of the drug and/or the number of hours to expire using the human interface device 1708. Further, selecting the number of hours to expire allow the nurse to simply handwrite a name of the drug on the one or more labels.

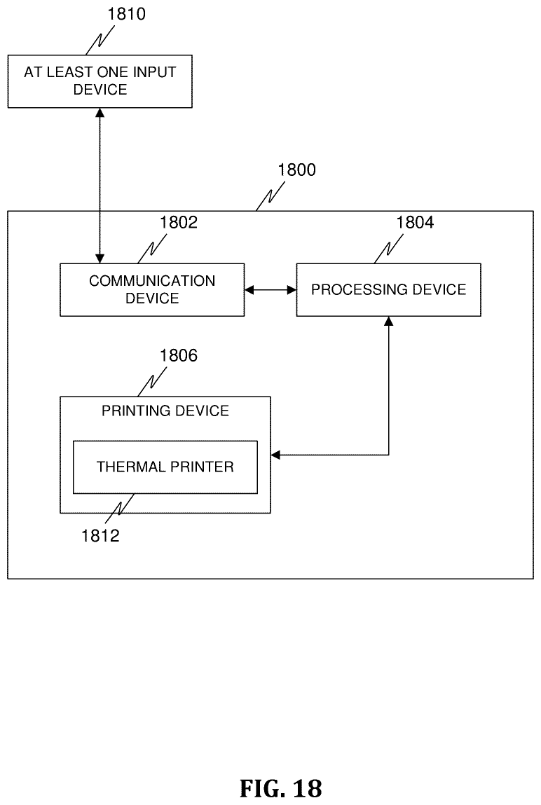

FIG. 18 is a block diagram of a system 1800 for creating labels for labeling intravenous infusion lines, in accordance with some embodiments. Accordingly, the system 1800 may include a communication device 1802 which may be configured for receiving one or more information associated with one or more intravenous infusion lines from one or more input devices 1810. Further, the system 1800 may include a processing device 1804 communicatively coupled with the communication device 1802. Further, the processing device 1804 may be configured for analyzing the one or more information. Further, the processing device 1804 may be configured for determining one or more label contents of one or more labels associated with the one or more intravenous infusion lines based on the analyzing. Further, the processing device 1804 may be configured for determining one or more label content formats of the one or more label contents based on the determining of the one or more label contents. Further, the one or more label content formats includes a color code. Further, the color code includes seven different colors corresponding to seven days of a calendar week. Further, the processing device 1804 may be configured for generating one or more label information for the one or more labels based on the one or more label contents and the one or more label content formats. Further, the system 1800 may include a printing device 1806 communicatively coupled with the processing device 1804. Further, the printing device 1806 may be configured for printing the one or more label information on one or more label sheets 1900 based on the generating of the one or more label information. Further, the printing device 1806 includes a thermal printer 1812. Further, the one or more label sheets 1900 includes a base substrate 1902 with a thermochromic coating 1908. Further, the thermochromic coating 1908 may be associated with two or more thermochromic compositions. Further, the one or more label sheets 1900 includes two or more sections. Further, the printing of the one or more label information further includes thermal printing of a required tubing change time alert on a section of the two or more sections. Further, the thermal printer 1812 may be configured for selectively inducing one or more chromatic transitions in one or more thermochromic compositions of the two or more thermochromic compositions. Further, the thermal printing of the required tubing change time alert on the section may be further based on the selective induction of the one or more chromatic transitions. Further, the thermal printing in the section color codes the required tubing change time alert with a color from the seven different colors representing a day of the seven days corresponding to the required tubing change time alert. Further, the printing of the one or more label information creates the one or more labels for the one or more intravenous infusion lines.

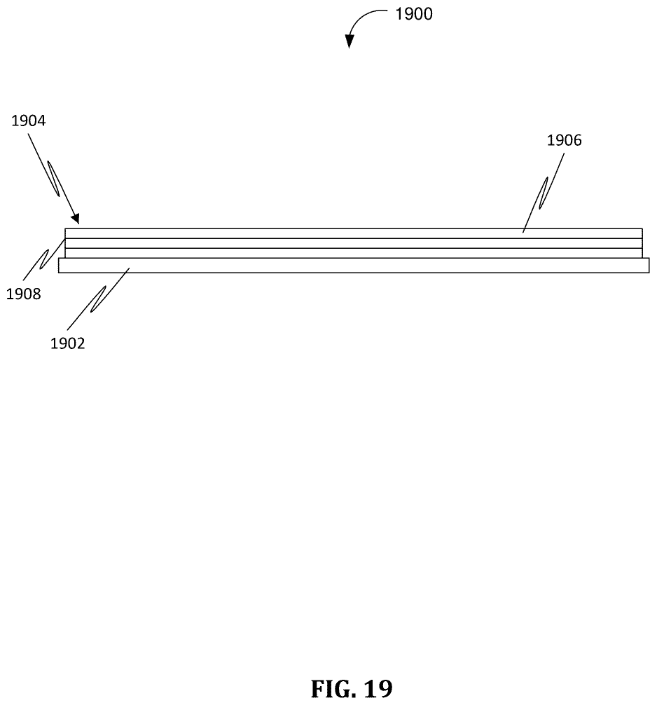

FIG. 19 illustrates selective heating of one or more thermochromic layers 1906 from two or more thermochromic layers 1904 relative to one or more label sheets 1900 in accordance with some embodiments. Further, in some embodiments, the one or more label sheets 1900 further include an adhesive layer which may be configured to be positioned in association with the base substrate 1902. Further, the thermochromic coating 1908 includes the two or more thermochromic layers 1904. Further, each of the two or more thermochromic layers 1904 may be associated with one or more of the two or more thermochromic compositions. Further, the thermal printer 1812 may be further configured for selectively heating the one or more thermochromic layers 1906 from the two or more thermochromic layers 1904 of the thermochromic coating 1908. Further, the thermal printing of the required tubing change time alert on the section may be further based on the selective heating of the one or more thermochromic layers 1906.

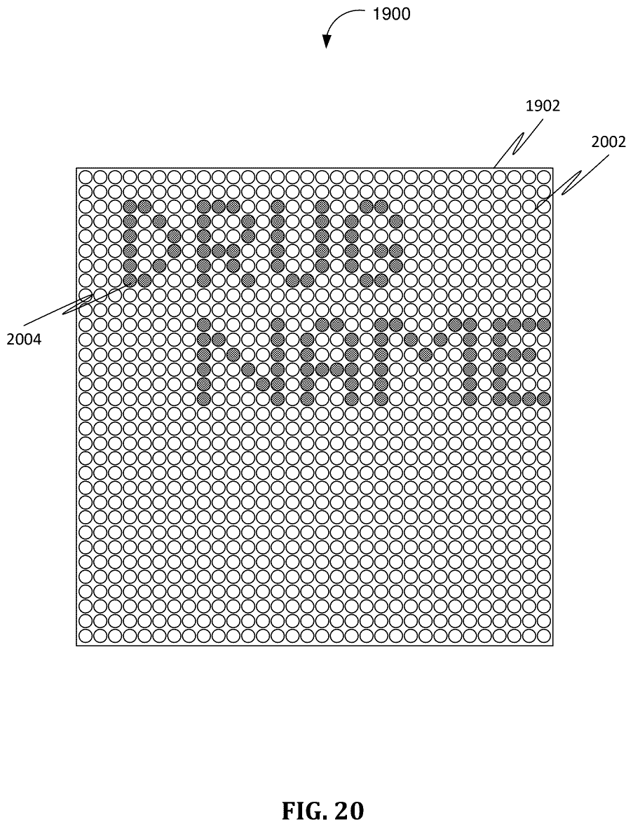

FIG. 20 illustrates selective heating of the one or more thermochromic regions 2004 from the two or more thermochromic regions 2002 relative to one or more label sheets 1900 in accordance with some embodiments. Further, the one or more label sheets 1900 includes the base substrate 1902 with the thermochromic coating 1908 comprising the two or more thermochromic regions 2002.

In some embodiments, the thermochromic coating 1908 includes two or more thermochromic regions 2002. Further, each of the two or more thermochromic regions 2002 may be associated with one or more of the two or more thermochromic compositions. Further, the thermal printer 1812 may be configured for selectively heating one or more thermochromic regions 2004 from the two or more thermochromic regions 2002. Further, the thermal printing of the required tubing change time alert on the section may be further based on the selective heating of the one or more thermochromic regions 2004.

In some embodiments, the processing device 1804 may be further configured for determining one or more operating parameters associated with the thermal printer 1812 based on the one or more label information. Further, the thermal printer 1812 may be further configured to be operated using the one or more operating parameters. Further, the selective induction of the one or more chromatic transitions in the one or more thermochromic compositions may be further based on the operating of the thermal printer 1812 using the one or more operating parameters.

In some embodiments, the one or more operating parameters includes one or more controlled temperatures. Further, the operating of the thermal printer 1812 using the one or more operating parameters includes applying the one or more controlled temperatures. Further, the selective induction of the one or more chromatic transitions in the one or more thermochromic compositions based on the operating of the thermal printer 1812 using the one or more operating parameters facilitates activation of one or more colors associated with the one or more thermochromic compositions. Further, the applying of the one or more controlled temperatures includes applying one or more of a standard temperature of sixty-five degrees Celsius, a low temperature within a range of forty degrees Celsius to forty-five degrees Celsius, a very low temperature within a range of thirty degrees Celsius to thirty-five degrees Celsius, and a high temperature within a range of seventy-five degrees Celsius to eighty degrees Celsius. Further, the applying of the standard temperature facilitates the activation of a black color in the one or more thermochromic compositions. Further, the applying of the low temperature facilitates the activation of a blue color in the one or more thermochromic compositions. Further, the applying of the very low temperature facilitates the activation of a yellow color in the one or more thermochromic compositions. Further, the applying of the high temperature facilitates the activation of an orange color in the one or more thermochromic compositions.

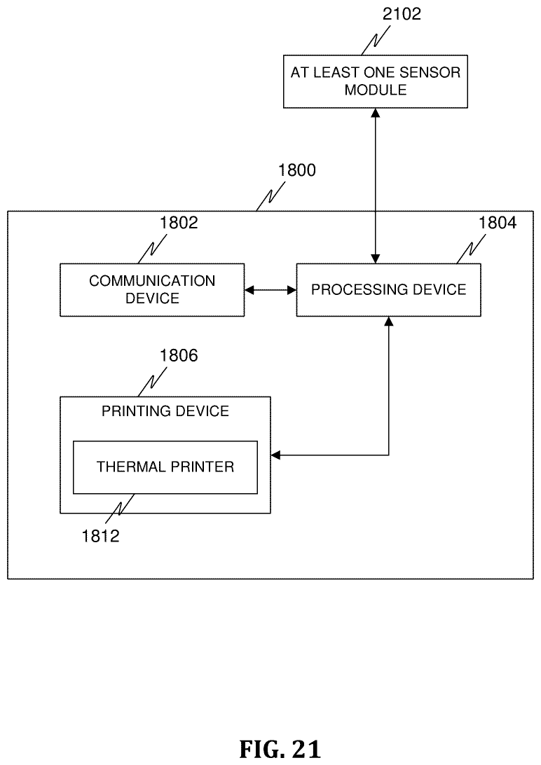

FIG. 21 illustrates at least one sensor module 2102 included in the system 1800 for creating labels for labeling intravenous infusion lines, in accordance with some embodiments. Further, in some embodiments, the selective heating of the one or more thermochromic regions 2004 may be associated with one or more temperature change relatives to the thermochromic coating 1908. Further, the system 1800 further includes the one or more sensor modules 2102 communicatively coupled with the processor. Further, the one or more sensor modules 2102 may be configured to be positioned proximal to the one or more label sheets 1900. Further, the one or more sensor modules 2102 may be further configured for detecting the one or more temperature changes. Further, the determining of the one or more operating parameters for the thermal printer 1812 may be further based on the detecting of the one or more temperature changes.

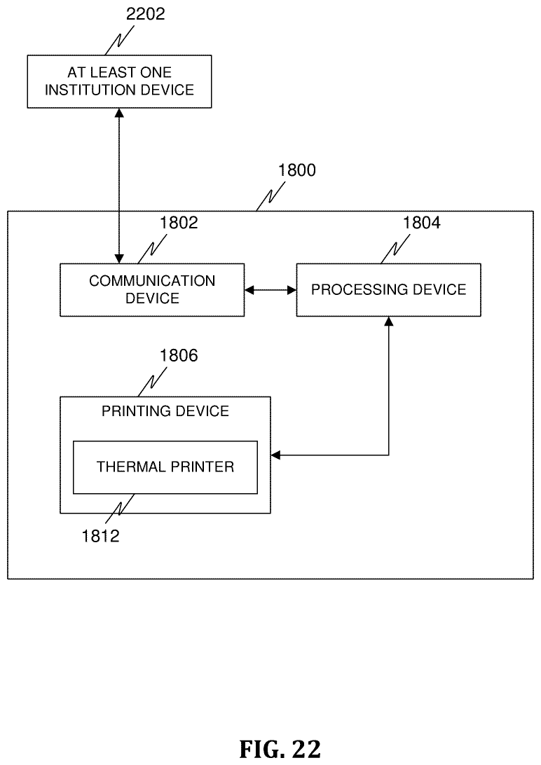

FIG. 22 illustrates at least one institution device 2202 included in the system 1800 for creating labels for labeling intravenous infusion lines, in accordance with some embodiments. Further, in some embodiments, the communication device 1802 may be further configured for receiving a color palette information from one or more institution devices 2202 associated with one or more institutions. Further, the color palette information representing one or more color palettes associated with the one or more institutions. Further, the processing device 1804 may be further configured for analyzing the one or more color palette information. Further, the generating of the one or more label information may be further based on the analyzing of the one or more color palette information.

In some embodiments, the one or more operating parameters further includes a heat application duration corresponding to a duration associated with the selective heating of the one or more thermochromic layers 1906. Further, the operating of the thermal printer 1812 using the one or more operating parameters includes controlling the heat application duration. Further, the one or more thermochromic layers 1906 includes a first layer and a second layer. Further, the second layer may be configured to be positioned below the first layer. Further, the heat application duration includes one or more of a short heat application duration and a long heat application duration. Further, the selective heating of the one or more thermochromic layers 1906 includes the selective heating of the one or more thermochromic layers 1906 based on the operating of the thermal printer 1812 using the one or more operating parameters. Further, the selective heating of the one or more thermochromic layers 1906 based on the operating of the thermal printer 1812 using the one or more operating parameters includes the selective heating of the first layer based on the short heat application duration and the selective heating of the second layer based on the long heat application duration.

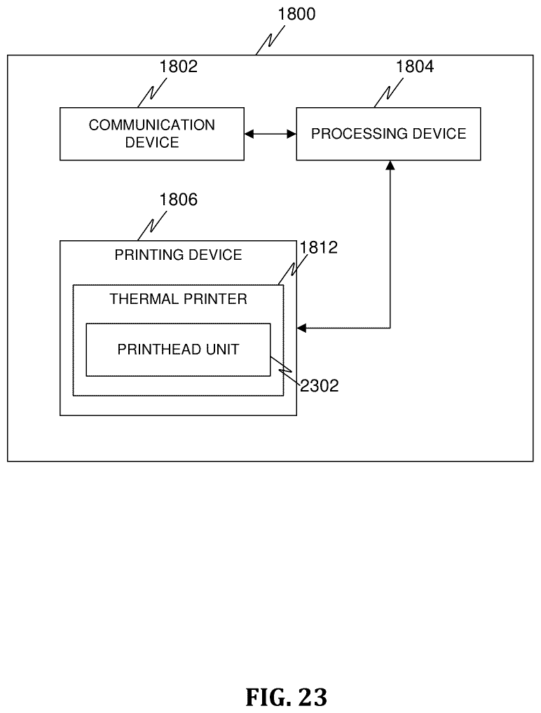

FIG. 23 illustrates a printhead unit 2302 included in the system 1800 for creating labels for labeling intravenous infusion lines, in accordance with some embodiments. Further, in some embodiments, the thermal printer 1812 includes the printhead unit 2302 which may be configured for facilitating the thermal printing of the required tubing change time alert. Further, the one or more operating parameters includes one or more controlled pressures corresponding to a pressure applied by the printhead unit 2302 on the one or more label sheets 1900. Further, the operating of the thermal printer 1812 using the one or more operating parameters includes controlling the one or more controlled pressures. Further, the selective induction of the one or more chromatic transitions in the one or more thermochromic compositions may be further based on the controlling of the one or more controlled pressures.

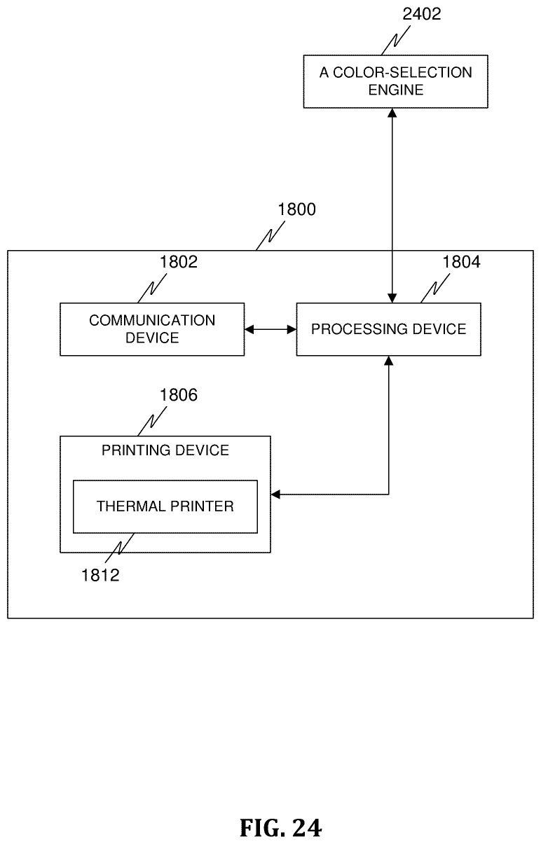

FIG. 24 illustrates a color-selection engine 2402 included in the system 1800 for creating labels for labeling intravenous infusion lines, in accordance with some embodiments. Further, in some embodiments, the processing device 1804 may be further configured for identifying one or more colors from two or more colors using a color-selection engine 2402 based on the one or more label information. Further, the processing device 1804 may be further configured for determining one or more print-paths for the one or more colors using a print-path planning algorithm based on the identifying of the one or more colors. Further, the printing of the one or more label information on the one or more label sheets 1900 may be further based on the determining of the one or more print-paths.

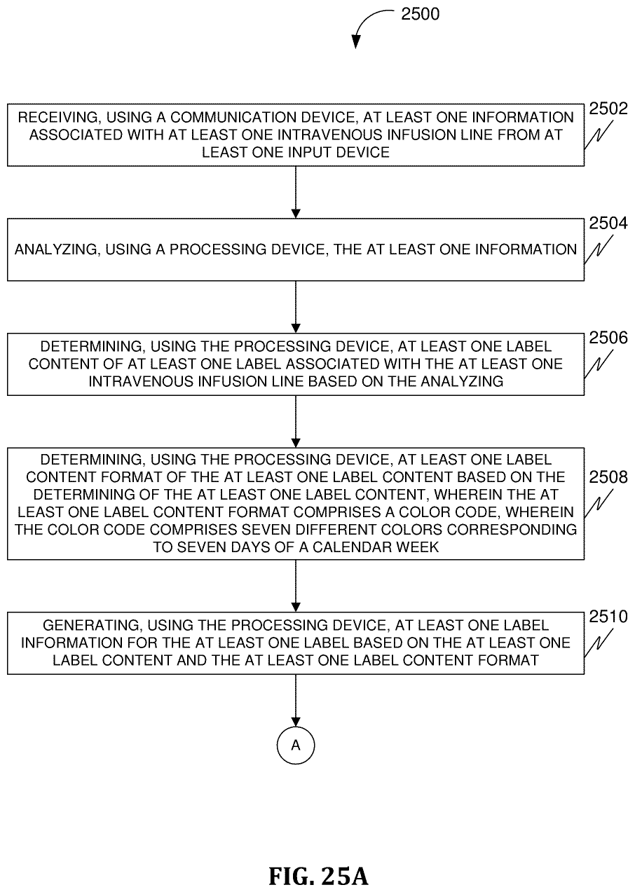

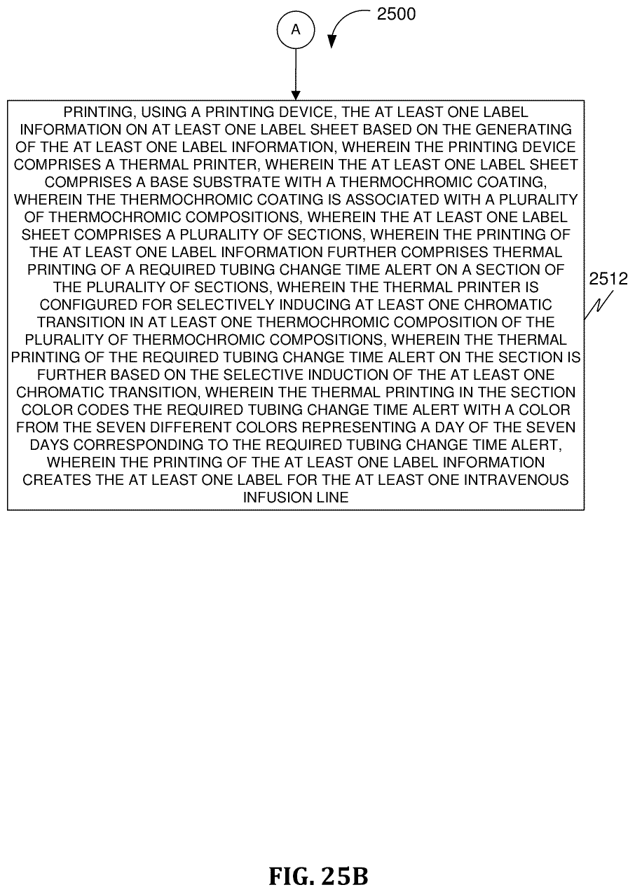

FIG. 25A and FIG. 25B illustrate a flowchart of a method 2500 for creating a label for labeling an intravenous infusion line, in accordance with some embodiments. Accordingly, the method 2500 may include a step 2502 of receiving, using a communication device 1802, one or more information associated with one or more intravenous infusion lines from one or more input devices 1810. Further, the method 2500 may include a step 2504 of analyzing, using a processing device 1804, the one or more information. Further, the method 2500 may include a step 2506 of determining, using the processing device 1804, one or more label contents of one or more labels associated with the one or more intravenous infusion lines based on the analyzing. Further, the method 2500 may include a step 2508 of determining, using the processing device 1804, one or more label content formats of the one or more label contents based on the determining of the one or more label contents. Further, the one or more label content formats includes a color code. Further, the color code includes seven different colors corresponding to seven days of a calendar week. Further, the method 2500 may include a step 2510 of generating, using the processing device 1804, one or more label information for the one or more labels based on the one or more label contents and the one or more label content formats. Further, the method 2500 may include a step 2512 of printing, using a printing device 1806, the one or more label information on one or more label sheets 1900 based on the generating of the one or more label information. Further, the printing device 1806 includes a thermal printer 1812. Further, the one or more label sheets 1900 includes a base substrate 1902 with a thermochromic coating 1908. Further, the thermochromic coating 1908 may be associated with two or more thermochromic compositions. Further, the one or more label sheets 1900 includes two or more sections. Further, the printing of the one or more label information further includes thermal printing of a required tubing change time alert on a section of the two or more sections. Further, the thermal printer 1812 may be configured for selectively inducing one or more chromatic transitions in one or more thermochromic compositions of the two or more thermochromic compositions. Further, the thermal printing of the required tubing change time alert on the section may be further based on the selective induction of the one or more chromatic transitions. Further, the thermal printing in the section color codes the required tubing change time alert with a color from the seven different colors representing a day of the seven days corresponding to the required tubing change time alert. Further, the printing of the one or more label information creates the one or more labels for the one or more intravenous infusion lines.

In some embodiments, the one or more label sheets 1900 further includes an adhesive layer which may be configured to be positioned in association with the base substrate 1902. Further, the thermochromic coating 1908 includes two or more thermochromic layers 1904. Further, each of the two or more thermochromic layers 1904 may be associated with one or more of the two or more thermochromic compositions. Further, the thermal printer 1812 may be further configured for selectively heating one or more thermochromic layers 1906 from the two or more thermochromic layers 1904 of the thermochromic coating 1908. Further, the thermal printing of the required tubing change time alert on the section may be further based on the selective heating of the one or more thermochromic layers 1906.

In some embodiments, the thermochromic coating 1908 includes two or more thermochromic regions 2002. Further, each of the two or more thermochromic regions 2002 may be associated with one or more of the two or more thermochromic compositions.

Further, the thermal printer 1812 may be configured for selectively heating one or more thermochromic regions 2004 from the two or more thermochromic regions 2002. Further, the thermal printing of the required tubing change time alert on the section may be further based on the selective heating of the one or more thermochromic regions 2004.

In some embodiments, the method may further include determining, using the processing device 1804, one or more operating parameters associated with the thermal printer 1812 based on the one or more label information. Further, the thermal printer 1812 may be further configured to be operated using the one or more operating parameters. Further, the selective induction of the one or more chromatic transitions in the one or more thermochromic compositions may be further based on the operating of the thermal printer 1812 using the one or more operating parameters.

In some embodiments, the one or more operating parameters includes one or more controlled temperatures. Further, the operating of the thermal printer 1812 using the one or more operating parameters includes applying the one or more controlled temperatures. Further, the selective induction of the one or more chromatic transitions in the one or more thermochromic compositions based on the operating of the thermal printer 1812 using the one or more operating parameters facilitates activation of one or more colors associated with the one or more thermochromic compositions. Further, the applying of the one or more controlled temperatures includes applying one or more of a standard temperature of sixty-five degrees Celsius, a low temperature within a range of forty degrees Celsius to forty-five degrees Celsius, a very low temperature within a range of thirty degrees Celsius to thirty-five degrees Celsius, and a high temperature within a range of seventy-five degrees Celsius to eighty degrees Celsius. Further, the applying of the standard temperature facilitates the activation of a black color in the one or more thermochromic compositions. Further, the applying of the low temperature facilitates the activation of a blue color in the one or more thermochromic compositions. Further, the applying of the very low temperature facilitates the activation of a yellow color in the one or more thermochromic compositions. Further, the applying of the high temperature facilitates the activation of an orange color in the one or more thermochromic compositions.

In some embodiments, the selective heating of the one or more thermochromic regions 2004 may be associated with one or more temperature change relatives to the thermochromic coating 1908. Further, the method 2500 further includes detecting, using one or more sensor modules 2102, the one or more temperature changes. Further, the determining of the one or more operating parameters for the thermal printer 1812 may be further based on the detecting of the one or more temperature changes.

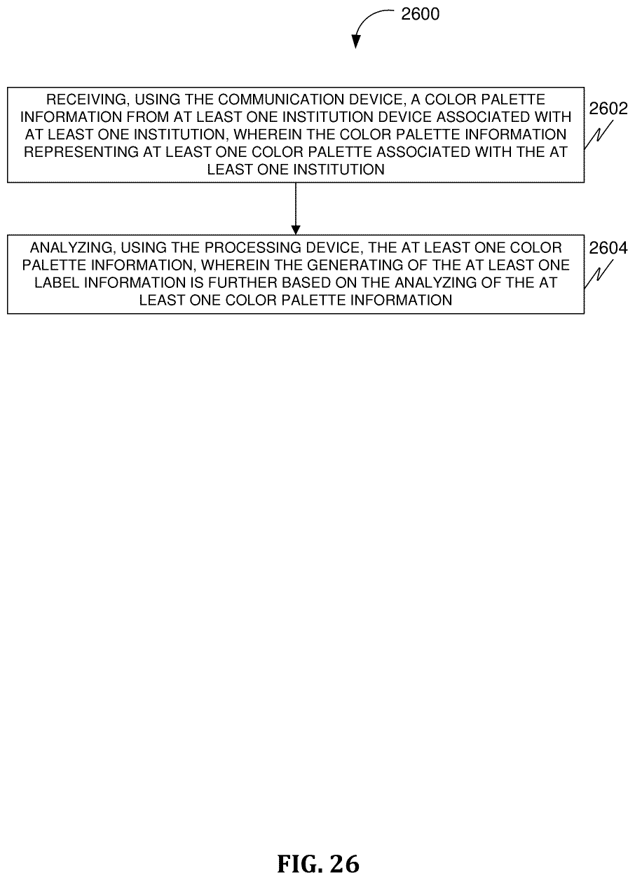

FIG. 26 illustrates a flowchart of a method 2600 for creating a label for labeling an intravenous infusion line including analyzing, using the processing device 1804, the at least one color palette information, in accordance with some embodiments. Further, in some embodiments, the method 2600 further may include a step 2602 of receiving, using the communication device 1802, a color palette information from one or more institution devices 2202 associated with one or more institutions. Further, the color palette information representing one or more color palettes associated with the one or more institutions. Further, in some embodiments, the method 2600 further may include a step 2604 of analyzing, using the processing device 1804, the one or more color palette information. Further, the generating of the one or more label information may be further based on the analyzing of the one or more color palette information.

In some embodiments, the one or more operating parameters further includes a heat application duration corresponding to a duration associated with the selective heating of the one or more thermochromic layers 1906. Further, the operating of the thermal printer 1812 using the one or more operating parameters includes controlling the heat application duration. Further, the one or more thermochromic layers 1906 includes a first layer and a second layer. Further, the second layer may be configured to be positioned below the first layer. Further, the heat application duration includes one or more of a short heat application duration and a long heat application duration. Further, the selective heating of the one or more thermochromic layers 1906 includes the selective heating of the one or more thermochromic layers 1906 based on the operating of the thermal printer 1812 using the one or more operating parameters. Further, the selective heating of the one or more thermochromic layers 1906 based on the operating of the thermal printer 1812 using the one or more operating parameters includes the selective heating of the first layer based on the short heat application duration and the selective heating of the second layer based on the long heat application duration.

In some embodiments, the thermal printer 1812 includes a printhead unit 2302 which may be configured for facilitating the thermal printing of the required tubing change time alert. Further, the one or more operating parameters includes one or more controlled pressures corresponding to a pressure applied by the printhead unit 2302 on the one or more label sheets 1900. Further, the operating of the thermal printer 1812 using the one or more operating parameters includes controlling the one or more controlled pressures. Further, the selective induction of the one or more chromatic transitions in the one or more thermochromic compositions may be further based on the controlling of the one or more controlled pressures.

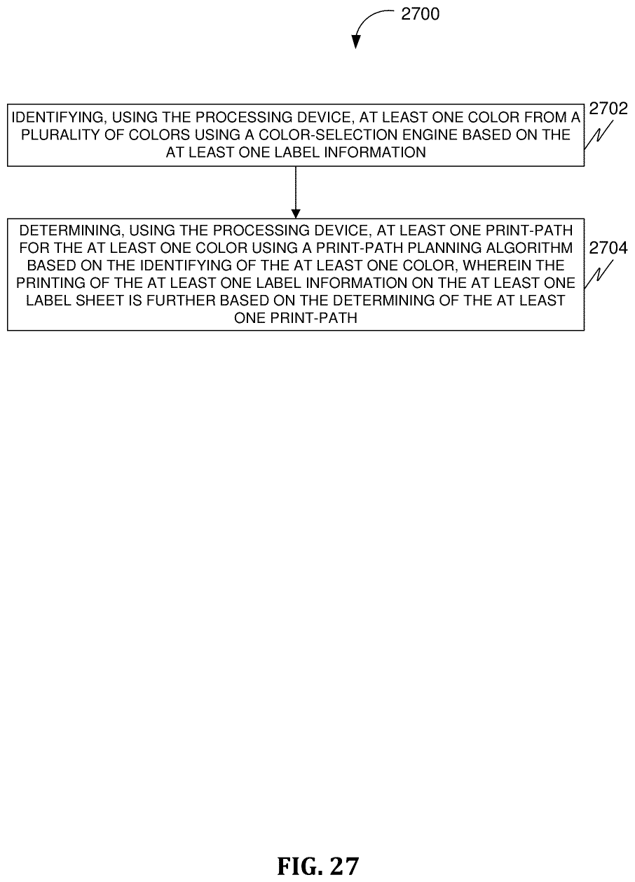

FIG. 27 illustrates a flowchart of a method 2700 for creating a label for labeling an intravenous infusion line including determining, using the processing device 2704, at least one print-path, in accordance with some embodiments. Further, in some embodiments, the method 2700 further may include a step 2702 of identifying, using the processing device 1804, one or more colors from two or more colors using a color-selection engine 2402 based on the one or more label information. Further, in some embodiments, the method 2700 further may include a step 2704 of determining, using the processing device 1804, one or more print-paths for the one or more colors using a print-path planning algorithm based on the identifying of the one or more colors. Further, the printing of the one or more label information on the one or more label sheets 1900 may be further based on the determining of the one or more print-paths.

In some embodiments, the two or more thermochromic regions 2002 includes two or more surface pixels. Further, the printhead unit 2302 includes two or more micro-heating elements. Further, the each of the two or more micro-heating elements may be configured for selectively heating one or more surface pixels from the two or more surface pixels. Further, the selective heating of the one or more thermochromic regions 2004 may be further based on the selective heating of the one or more surface pixels from the two or more surface pixels.

In some embodiments, the two or more thermochromic layers 1904 includes one or more of a first layer characterized by a black color, a second layer characterized by a blue color, a third layer characterized by a yellow color, and a fourth layer characterized by an orange color.

In some embodiments, the one or more operating parameters includes one or more of a temperature, a pressure, and a pulse duration associated with the thermal printer 1812 for thermal printing of the required tubing change time alert on the section.

In some embodiments, the one or more label information includes one or more of a day-of-week indicator, a drug-specific warning, the required tubing change time alert, and an infusion status icon associated with the one or more intravenous infusion lines.

In some embodiments, the printhead unit 2302 may be further configured for facilitating a heat-zone isolation on the one or more label sheets 1900. Further, the selective induction of the one or more chromatic transitions in the one or more thermochromic compositions may be further based on the heat-zone isolation.

In some embodiments, each of the two or more thermochromic layers 1904 may be characterized by a selective permeability. Further, the selective heating of the one or more thermochromic layers 1906 from the two or more thermochromic layers 1904 may be further based on the selective permeability.

In some embodiments, each of the two or more thermochromic layers 1904 may be configured to be spatially overlapping.

In some embodiments, the two or more sections includes one or more of a drug name section, a tubing change alert section, a day-of-week section, an infusion location section, a line type section, and a barcode/QR code region.

In some embodiments, the one or more sensor modules 2102 includes a feedback sensor module. Further, the feedback sensor module includes one or more of an infrared-based sensor module and a resistance-based sensor module.

In some embodiments, the activation of one or more of the red color and the orange color corresponds to an imminent tubing change. Further, the activation of the yellow color corresponds to a mid-window warning for tubing-change, may. Further, the activation of one or more of the blue color and the black color corresponds to one or more informational messages associated with the one or more intravenous infusion lines.

In some embodiments, the printing of the one or more label information further includes thermal printing of the one or more label information. Further, the one or more label information includes one or more of one or more permanent information and one or more temporary information. Further, the one or more thermochromic regions 2004 includes one or more of a first region associated with a reversible dye and a second region associated with an irreversible dye. Further, the thermal printing of the required tubing change time alert based on the selective induction of the one or more chromatic transitions in the one or more thermochromic compositions includes the thermal printing of the one or more permanent information based on the selective heating of the second region and the thermal printing of the one or more temporary information based on the selective heating of the first region.

In some embodiments, the selective heating of the one or more thermochromic regions 2004 includes exposing the one or more thermochromic regions 2004 to an infrared radiation (IR). Further, the thermal printing of the required tubing change time alert on the section may be further based on the exposing the one or more thermochromic regions 2004 to the IR.

In some embodiments, the one or more operating parameters includes a heating wavelength associated with the IR. Further, the operating of the thermal printer 1812 using the one or more operating parameters includes controlling the heating wavelength. Further, the exposing of the one or more thermochromic regions 2004 to the IR may be further based on the controlling of the heating wavelength.

In some embodiments, the thermochromic coating 1908 includes one or more conductive pathways which may be configured for directing a heat through the thermochromic coating 1908 during the thermal printing. Further, the selective heating of the one or more thermochromic regions 2004 from the two or more thermochromic regions 2002 may be further based on the directing of the heat through the thermochromic coating 1908.

In some embodiments, the thermochromic coating 1908 includes two or more magnetic nanoparticles. Further, the thermal printer 1812 may be further configured for facilitating a magnetic induction heating procedure. Further, the thermal printing of the one or more label information includes thermal printing of the one or more label information using the two or more magnetic nanoparticles based on the magnetic induction heating procedure.

In some embodiments, each of the two or more thermochromic layers 1904 may be associated with an adsorption characteristic. Further, the selective heating of the one or more thermochromic layers 1906 includes one or more of a light emitting diode (LED) based heating and a laser based heating of the one or more thermochromic layers 1906 based on the adsorption characteristic. Further, the thermal printing of the one or more label information may be further based on one or more of the LED based heating and the laser based heating.

In some embodiments, the base substrate 1902 may be one or more of a paper and a polymer film.

In some embodiments, the one or more temporary information includes an infusion status indicator. Further, the infusion status indicator includes one or more of an infusion paused indicator and an infusion resumed indicator.

In some embodiments, the one or more permanent information includes one or more of a drug name and a patient ID.

In some embodiments, the one or more labels includes a primary label and one or more secondary labels. Further, a primary label content of the primary label includes one or more of a user information, a medicine information, a time and date of administration, a required tubing change time for intravenous infusion, a drug-specific warning, and a location of the infusion's administration. Further, the one or more secondary labels may be configured for tagging the one or more intravenous infusion lines in one or more places of the one or more intravenous infusion lines. Further, a secondary label content of the one or more secondary labels includes a drug name and an administration location information. Further, the thermal printing of the one or more label information includes one or more of thermal printing the primary label content based on the activation of the blue color, thermal printing the secondary label content based on the activation of the black color, thermal printing an emergency bolus information based on the activation of the orange color, and thermal printing a continuous infusion information based on the activation of the yellow color.

In some embodiments, the one or more label information includes one or more drug class indicators. Further, the thermal printing of the one or more drug class indicators includes one or more of thermal printing an antibiotic indicator based on the activation of the yellow color, thermal printing a sedative indicator based on the activation of the blue color, thermal printing a vasopressor indicator based on the activation of the orange color, thermal printing a hazardous drug indicator based on the activation of the black color.

In some embodiments, the one or more label sheets 1900 may be a thermochromic label sheet which may be configured for undergoing one or more color changes based on a temperature variation in a system 1800 environment associated with the system 1800.

In some embodiments, the thermochromic coating 1908 further includes one or more micro-insulating layers. Further, the one or more micro-insulating layers may be configured to be disposed between the two or more thermochromic layers 1904.

In some embodiments, the one or more operating parameters includes a pulse-width modulation executed by the thermal printer 1812 relative to the thermochromic coating 1908. Further, the selective induction of the one or more chromatic transitions in the one or more thermochromic compositions may be further based on the executing of the pulse-width modulation.

In some embodiments, the present disclosure provides a label sheet for labeling an intravenous infusion line. Further, the label sheet may include a substrate. Further, the substrate includes an adhesive backing. Further, the label sheet may include two or more die-cut labels disposed on the substrate. Further, the two or more die-cut labels may include a primary label which may be configured to be adhered to the intravenous infusion line. Further, the primary label may be further configured for presenting an intravenous infusion information. Further, the two or more die-cut labels may include one or more line-tracing labels which may be configured to be adhered to the intravenous infusion line. Further, the one or more line-tracing labels may be further configured for facilitating a visual tracing of the intravenous infusion line. Further, the one or more line-tracing labels may be elongate characterized by a length and a width. Further, the length of the one or more line-tracing labels may be greater than the width of the one or more line-tracing labels. Further, the one or more line-tracing labels includes one or more longitudinal trace regions extending along at least a majority of the length of the one or more line-tracing labels. Further, the longitudinal trace region may be configured for presenting a selected color from a predefined medical color set comprising between three and seven distinct colors. Further, the selected color corresponds to a clinically meaningful attribute associated with the intravenous infusion line.

In some embodiments, the predefined medical color set includes exactly seven distinct colors corresponding to seven days of a calendar week.

In some embodiments, the clinically meaningful attribute includes a required tubing change day. Further, the selected color may be the same as a day-indicator color presented on the primary label.

In some embodiments, the clinically meaningful attribute includes a line type information, a drug class information, a tubing-change urgency window information, and an infusion status. Further, the line type information includes one or more of a primary and a secondary/piggyback.

In some embodiments, the one or more longitudinal trace regions includes a single continuous stripe extending along at least seventy percentage of the length of the one or more line-tracing labels.

In some embodiments, the one or more longitudinal trace regions includes a pair of parallel stripes extending along the length of the one or more line-tracing labels.

In some embodiments, the pair of parallel stripes includes a first stripe encoding a first clinically meaningful attribute and a second stripe encoding a second clinically meaningful attribute. Further, the first clinically meaningful attribute may be different from the second clinically meaningful attribute.

In some embodiments, the first stripe may be printed in a first color selected from the predefined medical color set and the second stripe may be printed in a second color selected from the predefined medical color set, thereby forming a two-stripe color code for differentiating among two or more concurrent infusion lines.

In some embodiments, the one or more line-tracing labels may be characterized by a length-to-width aspect ratio of at least five to one, optionally at least ten to one.

In some embodiments, the label sheet may further include two or more line-tracing labels which may be configured for placement at two or more locations along the intravenous infusion line. Further, the two or more locations includes at least two of a location proximal to a patient insertion site, a location proximal to an infusion pump, a location above the infusion pump, a location below the infusion pump, and a location proximal to a Y-port.

In some embodiments, the longitudinal trace region may be visually discernible when the line-tracing label may be applied in one or more of a longitudinal orientation along the intravenous infusion line, and a helical wrap around the intravenous infusion line.

In some embodiments, the longitudinal trace region may be rendered as a repeating pattern along the length of the one or more line-tracing labels. Further, the rendering of the longitudinal trace as the repeating pattern maintains a visual traceability when wrapped around intravenous infusion line.

In some embodiments, the one or more line-tracing labels further includes a non-color cue associated with the selected color. Further, the non-color cue includes one or more of a hatching, a micro text, an alphanumeric day marker, and a geometric icon for supporting a color-blind identification.

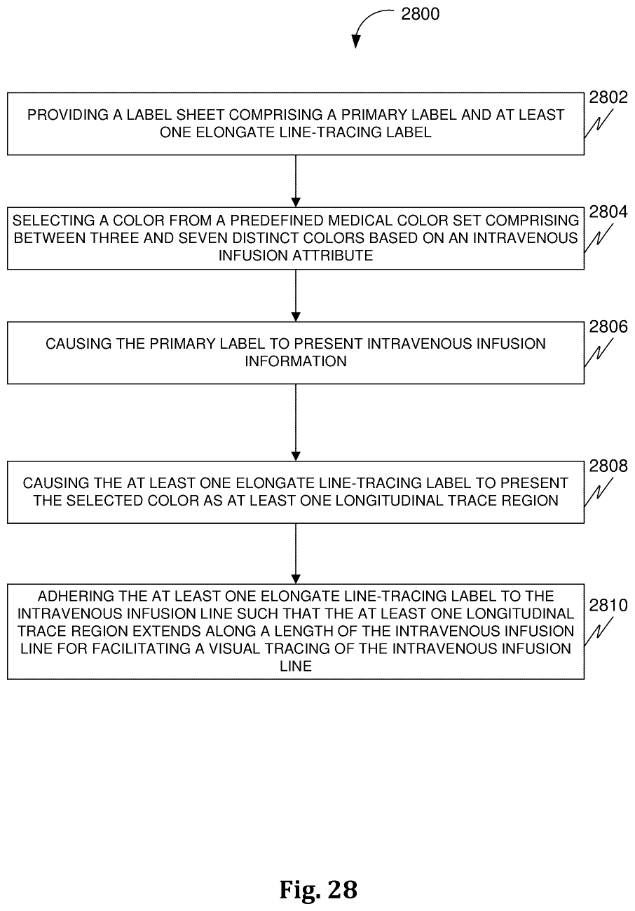

FIG. 28 illustrates a flowchart of a method 2800 for labeling an intravenous infusion line, in accordance with some embodiments. Further, the method 2800 may include a step 2802 of providing a label sheet comprising a primary label and one or more elongate line-tracing labels. Further, the method 2800 may include a step 2804 of selecting a color from a predefined medical color set comprising between three and seven distinct colors based on an intravenous infusion attribute. Further, the method 2800 may include a step 2806 of causing the primary label to present intravenous infusion information. Further, the method 2800 may include a step 2808 of causing the one or more elongate line-tracing labels to present the selected color as one or more longitudinal trace regions. Further, the method 2800 may include a step 2810 of adhering the one or more elongate line-tracing labels to the intravenous infusion line such that the one or more longitudinal trace regions extends along a length of the intravenous infusion line for facilitating a visual tracing of the intravenous infusion line.

In some embodiments, the adhering of the one or more elongate line-tracing labels to the intravenous infusion line includes applying a first line-tracing label above an infusion pump and applying a second line-tracing label below the infusion pump.

In some embodiments, the one or more longitudinal trace regions includes a pair of parallel stripes encoding two different infusion attributes.

In some embodiments, the predefined medical color set may be institution-configurable but constrained to between three and seven distinct colors.

In some embodiments, the one or more label sheets 1900 may include a primary label and one or more elongate line-tracing labels. Further, the one or more elongate line-tracing labels may include one or more longitudinal trace regions configured for presenting a selected color from a predefined medical color set comprising between three and seven distinct colors.

Further, the one or more longitudinal trace regions include a pair of parallel stripes.

Further, the selected color corresponds to a required tubing-change day. Further, the selected color matches a day-indicator color presented on the primary label.

Although the invention has been explained in relation to its preferred embodiment, it is to be understood that many other possible modifications and variations can be made without departing from the spirit and scope of the invention.

What is claimed is:

1. A system for creating a label for labeling an intravenous infusion line, the system comprising:
a communication device configured for receiving at least one information associated with at least one intravenous infusion line from at least one input device;
a processing device communicatively coupled with the communication device, wherein the processing device is configured for:
analyzing the at least one information;
determining at least one label content of at least one label associated with the at least one intravenous infusion line based on the analyzing;
determining at least one label content format of the at least one label content based on the determining of the at least one label content, wherein the at least one label content format comprises a color code, wherein the color code comprises seven different colors corresponding to seven days of a calendar week; and
generating at least one label information for the at least one label based on the at least one label content and the at least one label content format; and
a printing device communicatively coupled with the processing device, wherein the printing device is configured for printing the at least one label information on at least one label sheet based on the generating of the at least one label information, wherein the printing device comprises a thermal printer, wherein the at least one label sheet comprises a base substrate with a thermochromic coating, wherein the thermochromic coating is associated with a plurality of thermochromic compositions, wherein the at least one label sheet comprises a plurality of sections, wherein the printing of the at least one label information further comprises thermal printing of a required tubing change time alert on a section of the plurality of sections, wherein the thermal printer is configured for selectively inducing at least one chromatic transition in at least one thermochromic composition of the plurality of thermochromic compositions, wherein the thermal printing of the required tubing change time alert on the section is further based on the selective induction of the at least one chromatic transition, wherein the thermal printing in the section color codes the required tubing change time alert with a color from the seven different colors representing a day of the seven days corresponding to the required tubing change time alert, wherein the printing of the at least one label information creates the at least one label for the at least one intravenous infusion line.

2. The system of claim 1, wherein the at least one label sheet further comprises an adhesive layer configured to be positioned in association with the base substrate, wherein the thermochromic coating comprises a plurality of thermochromic layers, wherein each of the plurality of thermochromic layers is associated with at least one of the plurality of thermochromic compositions, wherein the thermal printer is further configured for selectively heating at least one thermochromic layer from the plurality of thermochromic layers of the thermochromic coating, wherein the thermal printing of the required tubing change time alert on the section is further based on the selective heating of the at least one thermochromic layer.

3. The system of claim 2, wherein the thermochromic coating comprises a plurality of thermochromic regions, wherein each of the plurality of thermochromic regions is associated with at least one of the plurality of thermochromic compositions, wherein the thermal printer is configured for selectively heating at least one thermochromic region from the plurality of thermochromic regions, wherein the thermal printing of the required tubing change time alert on the section is further based on the selective heating of the at least one thermochromic region.

4. The system of claim 3, wherein the processing device is further configured for determining at least one operating parameter associated with the thermal printer based on the at least one label information, wherein the thermal printer is further configured to be operated using the at least one operating parameter, wherein the selective induction of the at least one chromatic transition in the at least one thermochromic composition is further based on the operating of the thermal printer using the at least one operating parameter.

5. The system of claim 4, wherein the at least one operating parameter comprises at least one controlled temperature, wherein the operating of the thermal printer using the at least one operating parameter comprises applying the at least one controlled temperature, wherein the selective induction of the at least one chromatic transition in the at least one thermochromic composition based on the operating of the thermal printer using the at least one operating parameter facilitates activation of at least one color associated with the at least one thermochromic composition, wherein the applying of the at least one controlled temperature comprises applying at least one of a standard temperature of sixty-five degree Celsius, a low temperature within a range of forty degree Celsius to forty-five degree Celsius, a very low temperature within a range of thirty degree Celsius to thirty-five degree Celsius, and a high temperature within a range of seventy-five degree Celsius to eighty degree Celsius, wherein the applying of the standard temperature facilitates the activation of a black color in the at least one thermochromic composition, wherein the applying of the low temperature facilitates the activation of a blue color in the at least one thermochromic composition, wherein the applying of the very low temperature facilitates the activation of a yellow color in the at least one thermochromic composition, wherein the applying of the high temperature facilitates the activation of an orange color in the at least one thermochromic composition.

6. The system of claim 4, wherein the selective heating of the at least one thermochromic region is associated with at least one temperature change relative to the thermochromic coating, wherein the system further comprises at least one sensor module communicatively coupled with the processor, wherein the at least one sensor module is configured to be positioned proximal to the at least one label sheet, wherein the at least one sensor module is further configured for detecting the at least one temperature change, wherein the determining of the at least one operating parameter for the thermal printer is further based on the detecting of the at least one temperature change.

7. The system of claim 1, wherein the communication device is further configured for receiving a color palette information from at least one institution device associated with at least one institution, wherein the color palette information representing at least one color palette associated with the at least one institution, wherein the processing device is further configured for analyzing the at least one color palette information, wherein the generating of the at least one label information is further based on the analyzing of the at least one color palette information.

8. The system of claim 4, wherein the at least one operating parameter further comprises a heat application duration corresponding to a duration associated with the selective heating of the at least one thermochromic layer, wherein the operating of the thermal printer using the at least one operating parameter comprises controlling the heat application duration, wherein the at least one thermochromic layer comprises a first layer and a second layer, wherein the second layer is configured to be positioned below the first layer, wherein the heat application duration comprises at least one of a short heat application duration and a long heat application duration, wherein the selective heating of the at least one thermochromic layer comprises the selective heating of the at least one thermochromic layer based on the operating of the thermal printer using the at least one operating parameter, wherein the selective heating of the at least one thermochromic layer based on the operating of the thermal printer using the at least one operating parameter comprises the selective heating of the first layer based on the short heat application duration and the selective heating of the second layer based on the long heat application duration.

9. The system of claim 4, wherein the thermal printer comprises a printhead unit configured for facilitating the thermal printing of the required tubing change time alert, wherein the at least one operating parameter comprises at least one controlled pressure corresponding to a pressure applied by the printhead unit on the at least one label sheet, wherein the operating of the thermal printer using the at least one operating parameter comprises controlling the at least one controlled pressure, wherein the selective induction of the at least one chromatic transition in the at least one thermochromic composition is further based on the controlling of the at least one controlled pressure.

10. The system of claim 1, wherein the processing device is further configured for:
identifying at least one color from a plurality of colors using a color-selection engine based on the at least one label information; and
determining at least one print-path for the at least one color using a print-path planning algorithm based on the identifying of the at least one color, wherein the printing of the at least one label information on the at least one label sheet is further based on the determining of the at least one print-path.

11. A method for creating a label for labeling an intravenous infusion line, the method comprising:
receiving, using a communication device, at least one information associated with at least one intravenous infusion line from at least one input device;
analyzing, using a processing device, the at least one information;
determining, using the processing device, at least one label content of at least one label associated with the at least one intravenous infusion line based on the analyzing;
determining, using the processing device, at least one label content format of the at least one label content based on the determining of the at least one label content, wherein the at least one label content format comprises a color code, wherein the color code comprises seven different colors corresponding to seven days of a calendar week; and
generating, using the processing device, at least one label information for the at least one label based on the at least one label content and the at least one label content format; and
printing, using a printing device, the at least one label information on at least one label sheet based on the generating of the at least one label information, wherein the printing device comprises a thermal printer, wherein the at least one label sheet comprises a base substrate with a thermochromic coating, wherein the thermochromic coating is associated with a plurality of thermochromic compositions, wherein the at least one label sheet comprises a plurality of sections, wherein the printing of the at least one label information further comprises thermal printing of a required tubing change time alert on a section of the plurality of sections, wherein the thermal printer is configured for selectively inducing at least one chromatic transition in at least one thermochromic composition of the plurality of thermochromic compositions, wherein the thermal printing of the required tubing change time alert on the section is further based on the selective induction of the at least one chromatic transition, wherein the thermal printing in the section color codes the required tubing change time alert with a color from the seven different colors representing a day of the seven days corresponding to the required tubing change time alert, wherein the printing of the at least one label information creates the at least one label for the at least one intravenous infusion line.

12. The method of claim 11, wherein the at least one label sheet further comprises an adhesive layer configured to be positioned in association with the base substrate, wherein the thermochromic coating comprises a plurality of thermochromic layers, wherein each of the plurality of thermochromic layers is associated with at least one of the plurality of thermochromic compositions, wherein the thermal printer is further configured for selectively heating at least one thermochromic layer from the plurality of thermochromic layers of the thermochromic coating, wherein the thermal printing of the required tubing change time alert on the section is further based on the selective heating of the at least one thermochromic layer.

13. The method of claim 12, wherein the thermochromic coating comprises a plurality of thermochromic regions, wherein each of the plurality of thermochromic regions is associated with at least one of the plurality of thermochromic compositions, wherein the thermal printer is configured for selectively heating at least one thermochromic region from the plurality of thermochromic regions, wherein the thermal printing of the required tubing change time alert on the section is further based on the selective heating of the at least one thermochromic region.

14. The method of claim 13 further comprising determining, using the processing device, at least one operating parameter associated with the thermal printer based on the at least one label information, wherein the thermal printer is further configured to be operated using the at least one operating parameter, wherein the selective induction of the at least one chromatic transition in the at least one thermochromic composition is further based on the operating of the thermal printer using the at least one operating parameter.

15. The method of claim 14, wherein the at least one operating parameter comprises at least one controlled temperature, wherein the operating of the thermal printer using the at least one operating parameter comprises applying the at least one controlled temperature, wherein the selective induction of the at least one chromatic transition in the at least one thermochromic composition based on the operating of the thermal printer using the at least one operating parameter facilitates activation of at least one color associated with the at least one thermochromic composition, wherein the applying of the at least one controlled temperature comprises applying at least one of a standard temperature of sixty-five degree Celsius, a low temperature within a range of forty degree Celsius to forty-five degree Celsius, a very low temperature within a range of thirty degree Celsius to thirty-five degree Celsius, and a high temperature within a range of seventy-five degree Celsius to eighty degree Celsius, wherein the applying of the standard temperature facilitates the activation of a black color in the at least one thermochromic composition, wherein the applying of the low temperature facilitates the activation of a blue color in the at least one thermochromic composition, wherein the applying of the very low temperature facilitates the activation of a yellow color in the at least one thermochromic composition, wherein the applying of the high temperature facilitates the activation of an orange color in the at least one thermochromic composition.

16. The method of claim 14, wherein the selective heating of the at least one thermochromic region is associated with at least one temperature change relative to the thermochromic coating, wherein the method further comprises detecting, using at least one sensor module, the at least one temperature change, wherein the determining of the at least one operating parameter for the thermal printer is further based on the detecting of the at least one temperature change.

17. The method of claim 11 further comprising:
receiving, using the communication device, a color palette information from at least one institution device associated with at least one institution, wherein the color palette information representing at least one color palette associated with the at least one institution; and
analyzing, using the processing device, the at least one color palette information, wherein the generating of the at least one label information is further based on the analyzing of the at least one color palette information.

18. The method of claim 14, wherein the at least one operating parameter further comprises a heat application duration corresponding to a duration associated with the selective heating of the at least one thermochromic layer, wherein the operating of the thermal printer using the at least one operating parameter comprises controlling the heat application duration, wherein the at least one thermochromic layer comprises a first layer and a second layer, wherein the second layer is configured to be positioned below the first layer, wherein the heat application duration comprises at least one of a short heat application duration and a long heat application duration, wherein the selective heating of the at least one thermochromic layer comprises the selective heating of the at least one thermochromic layer based on the operating of the thermal printer using the at least one operating parameter, wherein the selective heating of the at least one thermochromic layer based on the operating of the thermal printer using the at least one operating parameter comprises the selective heating of the first layer based on the short heat application duration and the selective heating of the second layer based on the long heat application duration.

19. The method of claim 14, wherein the thermal printer comprises a printhead unit configured for facilitating the thermal printing of the required tubing change time alert, wherein the at least one operating parameter comprises at least one controlled pressure corresponding to a pressure applied by the printhead unit on the at least one label sheet, wherein the operating of the thermal printer using the at least one operating parameter comprises controlling the at least one controlled pressure, wherein the selective induction of the at least one chromatic transition in the at least one thermochromic composition is further based on the controlling of the at least one controlled pressure.

20. The method of claim 11 further comprising:
identifying, using the processing device, at least one color from a plurality of colors using a color-selection engine based on the at least one label information; and
determining, using the processing device, at least one print-path for the at least one color using a print-path planning algorithm based on the identifying of the at least one color, wherein the printing of the at least one label information on the at least one label sheet is further based on the determining of the at least one print-path.
